(19) United States
(12) Patent Application Publication
(10) Pub. No.: US 2026/0092313 A1
(43) Pub. Date: Apr. 2, 2026

(54) NUCLEIC ACID DETECTION METHODS

(71) Applicant: The Charles Stark Draper Laboratory, Inc., Cambridge, MA (US)

(72) Inventors: Isaac Moran, Cambridge, MA (US); Kim Kelly, Cambridge, MA (US); Mike Turo, Cambridge, MA (US); Erin Rosenberger, Cambridge, MA (US)

(73) Assignee: The Charles Stark Draper Laboratory, Inc., Cambridge, MA (US)

(21) Appl. No.: 19/294,190

(22) Filed: Aug. 7, 2025

Related U.S. Application Data
(60) Provisional application No. 63/680,595, filed on Aug. 7, 2024.

(51) Int. Cl. C12Q 1/6825; C12Q 1/6806; C12Q 1/6834

(52) U.S. Cl. CPC C12Q 1/6825; C12Q 1/6806; C12Q 1/6834

(57) ABSTRACT

The disclosure relates to methods and systems for the detection of low-quantity (e.g., 1×10² copies/μL) target nucleic acids (e.g., genomic RNA and/or genomic DNA).

### RELATED APPLICATIONS

This application claims the benefit under 35 U.S.C. § 119(e) of U.S. Provisional Patent Application No. 63/680,595 filed Aug. 7, 2024, entitled “NUCLEIC ACID DETECTION METHODS,” the entire contents of which are incorporated by reference herein.

### REFERENCE TO AN ELECTRONIC SEQUENCE LISTING

The content of the electronic sequence listing (C175670000US01-SEQ-MAT.xml; Size: 21,318 bytes; and Date of Creation: Aug. 7, 2025) is herein incorporated by reference in its entirety.

### BACKGROUND

Standard DNA microarrays with optical output cannot generate sufficient signal to detect target nucleic acids at concentrations below 1 nM. Previous attempts to address this issue using CRISPR based chemistry found that enzyme(s) were over-active when immobilized through grafted gRNA. gRNAs alone are not efficient enough to reach concentration ranges of interest. Furthermore, the RNA probes were more substantially plagued by specificity issues.

### SUMMARY

Presented herein are systems and methods that utilize surface chemistry to enable DNA microarrays to detect genomic nucleic acid targets, either RNA or DNA or a combination thereof, at low concentrations, in the range of 1×10² to 1×10³ copies/μL (e.g., to identify pathogens in patient samples). Such methods involve, in some embodiments, the use of advanced polymer surface coatings, a dual-probe target capture motif with multi-labeled sense oligonucleotides, capture probes comprising locked nucleotides, and highly sensitive detectable molecules (e.g., quantum dots).

The methods provided herein detect and in some instances measure genomic RNA and/or DNA extracted from one or more organisms. The methods directly capture, label, and detect original genomic RNA and/or DNA in a variety of samples. No conversion, alteration, or amplification by replication is required or performed in order to achieve the sensitivity reported herein. The working examples demonstrate the ability to detect individually or in combination human cells, bacteria and viruses, the latter of which typically present with RNA as their most abundant nucleic acid.

Accordingly, in some aspects, the disclosure provides a method of determining the concentration of a target nucleic acid in a sample, the method comprising (i) contacting a sample comprising the target nucleic acid with a sense oligonucleotide and a capture oligonucleotide that is immobilized to a surface via a silane-containing perfluorinated linker to produce a surface-immobilized target nucleic acid, wherein the capture oligonucleotide comprises a capture domain that is complementary to a first region of the target nucleic acid, wherein the sense oligonucleotide is functionalized with a first member of a binding pair and comprises a target-binding domain that is complementary to a second region of the target nucleic acid, and wherein the first region of the target nucleic acid does not overlap with the second region of the target nucleic acid; (ii) contacting the surface-immobilized target nucleic acid with a detectable molecule that is conjugated to a second member of the binding pair; and (iii) determining the concentration of the target nucleic acid based on detection of the detectable molecule.

Some aspects of the disclosure provide a method of detecting a target nucleic acid in a sample comprising: (i) contacting a sample in a reaction mixture with (a) a capture oligonucleotide that is immobilized to a surface via a silane-containing perfluorinated linker, wherein the capture oligonucleotide comprises a capture domain that is complementary to a first region of the target nucleic acid, and (b) a sense oligonucleotide that is functionalized with a first member of a binding pair and comprises a target-binding domain that is complementary to a second region of the target nucleic acid, wherein the first region of the target nucleic acid does not overlap with the second region of the target nucleic acid; (ii) optionally removing nucleic acids that are not bound to the capture oligonucleotide from the reaction mixture; (iii) adding a detectable molecule that is conjugated to a second member of the binding pair to the reaction mixture; and (iv) detecting signal from surface-bound detectable label, wherein surface-bound signal at a level above background noise is indicative of the presence of the target nucleic acid in the sample. In some embodiments, surface-bound signal at a level at or below background noise is indicative of the absence of the target nucleic acid in the sample.

In some embodiments, the target nucleic acid is a ribonucleic acid (RNA). In some embodiments, the capture domain and/or the target-binding domain comprises one or more locked nucleotides (LNAs). In some embodiments, the silane-containing perfluorinated linker comprises the structure of Formula (I):

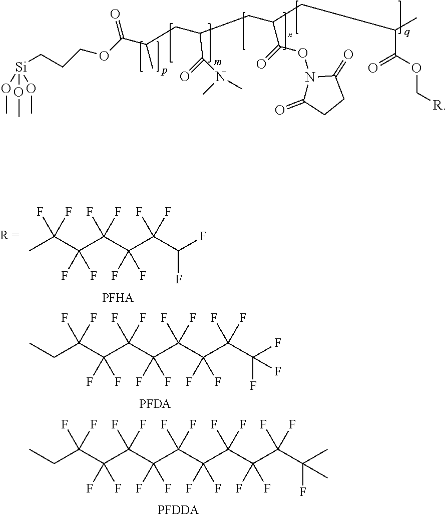

In some embodiments, the surface is a glass surface and may have a form-factor of a flat surface (such as a slide), a bead, a particle, and the like. In some embodiments, the surface is a silicon, silicon oxide, silicon nitride, polypropylene, or polystyrene surface. In some embodiments, the detectable molecule is a fluorescent molecule. In some embodiments, the fluorescent molecule is a fluorophore or a quantum dot. In some embodiments, the first member of the binding pair is biotin and/or the second member of the binding pair is streptavidin.

Other aspects and embodiments of the invention are described herein.

### BRIEF DESCRIPTION OF THE DRAWINGS

FIG. 1 shows an exemplary schematic of a method of the disclosure. The Figure illustrates capture of a target nucleic acid (e.g., RNA extracted from an organism) using a capture oligonucleotide immobilized to a surface and a “sense” oligonucleotide that is biotinylated (preferably in multiple locations along its length), followed by labeling of the sense oligonucleotide with detectable labels (e.g., quantum dots (QD)) that are conjugated to avidin or streptavidin and thus are able to bind to the biotin moieties. In some embodiments, the capture and sense oligonucleotides may comprise locked nucleotides as illustrated in FIG. 3.

FIG. 2 shows an exemplary linker structure for immobilization of a capture oligonucleotide to a surface (Lucidant Polymers, Sunnyvale, CA). The circled moiety is an NHS group for reacting with NH₂-functionalized oligonucleotides. The perfluoro groups represented by R control spot spreading and molecular adsorption.

FIG. 3 provides an exemplary structure of a locked nucleotide (circled moiety) as well as the structure of a sandwich assay involving the capture oligonucleotide, target, and sense oligonucleotide. In some embodiments, the capture oligonucleotide and the sense oligonucleotide each comprise 5-6 LNA.

FIG. 4 shows results obtained using an exemplary method of the disclosure. The top panel shows the results using a direct labeling method in which the target is biotinylated and then subsequently labeled with streptavidin-conjugated quantum dots. (The bar representing BH at 97.31×10³ copies per microliter has a value of 2326.) While sensitive, this method is cumbersome as it requires biotinylation (or other form of labeling) of the target. The bottom panel shows the results using a sandwich assay, as described in FIG. 3. (The bar representing BH at 126×10³ copies per microliter has a value of 891.) The target is a nucleic acid from bacteria Bordetella holmesii (BH). Target is detected with as few as about 5000 copies per microliter. For comparison, a SARS-CoV-2 patient presents with a respective target concentration of about 3,000-12,000 copies per microliter. Total assay time is about 1 hour. Insets in both top and bottom panel represent raw data from the assay when performed on a slide. Controls were capture oligonucleotides specific for 18S (human biomarker) and Flu virus (Flu H1N1, M gene, optionally denoted herein as Flu or FluM01 in the Figures). In each triplet of bars, the first bar is 18S, the second bar is BH, and the third bar is Flu H1N1, M gene.

FIG. 5 shows results obtained using an exemplary method of the disclosure. The data compare results from slides printed with capture oligonucleotides at 70% relative humidity (RH) (first 6 bars) and at 40% RH (last 6 bars). For each RH level, two target concentrations were tested (12.6×10³ copies per microliter and 126×10³ copies per microliter). Controls were capture oligonucleotides specific for 18S and Flu virus (Flu H1N1, M gene). In each triplet of bars, the first bar is 18S, the second bar is BH, and the third bar is Flu H1N1, M gene. The signal observed at 70% RH for 12.6×10³ copies per microliter is about 3-fold higher than the signal achieved in earlier experiments using an uncontrolled room humidity of about 30%. The follow-on experiments used a 70% RH condition.

FIG. 6 shows results obtained to study buffer conditions. The experiment tested two buffers: (1) 5×SSC+0.1% Tween (bottom panel, first 6 bars) and (2) MgCl₂+Tris-HCl (bottom panel, last 6 bars). The 5×SSC+0.1% Tween buffer yielded better signal, resulting in a 7× increase over the signal achieved in earlier experiments using 30% RH and 3×SSC as the buffer. This signal is above background (defined as 3×STD and illustrated by the dashed line). In each triplet of bars, the first bar is 18S, the second bar is BH, and the third bar is Flu H1N1, M gene. In the top panel, three target concentrations were tested (5.04×10³ copies per microliter, 12.60×10³ copies per microliter, and 126×10³ copies per microliter). In the bottom panel, two target concentrations were tested for each buffer type (5.04×10³ copies per microliter and 8.40×10³ copies per microliter). (The bar representing BH at 126×10³ copies per microliter in the top panel has a value of 891.) Note the difference in signal at 5.04×10³ copies per microliter in the top and bottom panels as evidence of the improved effect of the 5×SSC+0.1% Tween buffer.

FIG. 7 shows results obtained using sense oligonucleotides functionalized with a first member of a binding pair (e.g., biotin). Using biotinylated sense oligonucleotides improved signal compared to the direct binding assay of FIG. 4 (top panel). It also simplified the process and reduced assay time. An 11× signal increase was observed over the signal achieved in earlier experiments (and as exemplified in FIG. 4, bottom panel, sandwich assay). In each triplet of bars, the first bar is 18S, the second bar is BH, and the third bar is Flu H1N1, M gene. Two target concentrations were tested (50×10³ copies per microliter (first 3 bars) and 500×10³ copies per microliter (second 3 bars)).

FIG. 8 shows a compilation of results from multiple experiments obtained when factors were tested alone or in combination. Using 70% RH to print the capture oligonucleotide, 5×SSC+0.1% Tween as the buffer, and the biotinylated and locked nucleotide sense oligonucleotide described in FIG. 7, a 60× signal increase was observed over the signal achieved in initial experiments that used ˜30% RH, locked capture oligonucleotides, biotin functionalized sense oligonucleotides, and 3×SSC as the buffer for 1×10³ copies per microliter, and a 50× signal increase was observed over signal achieved in those initial experiments for 10×10³ copies per microliter. Two target concentrations were tested (1×10³ copies per microliter (first 3 bars: initial data, buffer, and new sense oligonucleotide) and 10×10³ copies per microliter (second 4 bars: initial data, print quality, buffer, and new sense oligonucleotide)).

FIG. 9 shows results obtained during experiments performed to determine a limit of detection for a specific target nucleic acid. The limit of detection (LOD) was found to be 500 copies per microliter of a target BH, which represents detection in the attomolar range. The BH 50×10³ copies per microliter bar has a value of 2770. Four target concentrations were tested (0.05×10³ copies per microliter (first 3 bars), 0.5×10³ copies per microliter (second 3 bars), 5.0×10³ copies per microliter (third 3 bars) and 50.0×10³ copies per microliter (fourth 3 bars)). In each triplet of bars, the first bar is 18S, the second bar is BH, and the third bar is Flu H1N1, M gene.

FIG. 10A shows results obtained using 18S nucleic acid as the target. In each triplet of bars, the first bar is 18S, the second bar is BH, and the third bar is Flu H1N1, M gene. Three target concentrations were tested (50,000×10³ copies per microliter (well 2), 5,000×10³ copies per microliter (well 3), and 500×10³ copies per microliter (well 4)).

FIG. 10B shows results obtained using a virus MS2 nucleic acid as the target. In each triplet of bars, the first bar is 18S, the second bar is BH, and the third bar is MS2 (virus). Four target concentration were tested (5,000,000×10³ copies per microliter (well 1), 500,000×10³ copies per microliter (well 2), 50,000×10³ copies per microliter (well 3), and 5,000×10³ copies per microliter (well 4)).

FIG. 10C shows results obtained using bacteria Legionella pneumophila (LP) nucleic acid as the target. In each triplet of bars, the first bar is 18S, the second bar is BH, and the third bar is LP. Four target concentrations were tested (350,000 copies per microliter (well 1), 50,000 copies per microliter (well 2), 10,000 copies per microliter (well 3), and 5,000 copies per microliter (well 4)).

FIG. 10D shows results obtained during multiplexing experiments in which 18S and BH target nucleic acids are detected. The results indicate little cross-talk between oligonucleotides for different targets and particularly no sense oligonucleotide interference. In each triplet of bars, the first bar is 18S, the second bar is BH, and the third bar is Flu H1N1, M gene. Four target concentration combinations were tested (0×10³ 18S copies per microliter and 50.0×10³ BH copies per microliter (well 1), 100,000×10³ 18S copies per microliter and 0×10³ BH copies per microliter (well 2), 100,000×10³ 18S copies per microliter and BH 50×10³ copies per microliter (well 3), and 18S 20,000×10³ copies per microliter and BH 10×10³ copies per microliter (well 4)).

FIG. 10E shows results obtained during multiplexing experiments in which human 18S, bacteria BH and virus MS2 target nucleic acids are detected. The results indicate little to no cross-talk between oligonucleotides for different targets including no sense oligonucleotide interference. The assay demonstrates orthogonality between multiple oligonucleotides (e.g., 3 capture oligonucleotides and 3 sense oligonucleotides for 3 different targets), and supports greater multiplexing capability. In each triplet of bars, the first bar is 18S, the second bar is BH, and the third bar is MS2. Well 1 shows the results of the experiment conducted with 18S nucleic acid as the target. Well 2 shows the results of the experiment conducted with BH nucleic acid as the target. Well 3 shows the results of the experiment conducted with MS2 nucleic acid as the target. Well 4 shows the results of the experiment conducted with 18S and BH nucleic acids as the targets. Well 5 shows the results of the experiment conducted with 18S and MS2 nucleic acids as the targets. Well 6 shows the results of the experiment conducted with BH and MS2 nucleic acids as the targets. Wells 7 and 8 show the results of experiments conducted with 18S, BH and MS2 nucleic acids as the targets. Well 8 used ⅕^{th} the amount of target as well 7, thereby showing that the multiplexed assay is sensitive and reproducible with differing (including significantly lower) amounts of target.

FIG. 11 shows the results obtained by performing the experiment using automated equipment on a sample that contains both 18S RNA (provided in the form of artificial nasal matrix (ANM) in the presence of viral transport medium (VTM)) and MS2 RNA. No cross-talk was observed between the oligonucleotides used to detect 18S, MS2 and BH nucleic acids.

FIG. 12 shows the results obtained by performing the experiment using automated equipment on a sample that contains both 18S RNA (provided in the form of ANM in the presence of VTM) and MS2 RNA (provided in the form of an MS2 bacteriophage).

FIG. 13 shows the results obtained by performing the experiment using a manual (slide) or an automated process (device) to detect MS2 target RNA. Using the manual process, MS2 target RNA was detected at concentrations of 5×10⁸ copies per microliter and 5×10⁷ copies per microliter, but not at lower copy numbers. Using the automated process, MS2 target RNA was detected at a concentration of 2×10⁴ PFU per microliter.

FIG. 14 shows the results obtained by performing the experiment using automated equipment on a swab collection sample that contains 18S RNA (provided in the form of ANM in the presence of VTM). No cross-talk was observed between the oligonucleotides used to detect 18S, BH and MS2 nucleic acids.

FIG. 15 shows a detection system for target nucleic acids, according to some embodiments.

FIG. 16A shows an example process flow for a method of detecting a target nucleic acid in a sample, according to some embodiments.

FIG. 16B shows an example process flow for a method of determining the concentration of a target nucleic acid in a sample, according to some embodiments.

FIG. 17 shows an exemplary block diagram of a special purpose computing system that may implement and/or execute methods described herein.

### DETAILED DESCRIPTION

Some aspects of the present disclosure relate to methods for the detection of low quantities of nucleic acids (e.g., as low as 1×10² nucleic acid copies per microliter). Provided herein are methods of detecting nucleic acids (and determining nucleic acid concentrations) in samples (e.g., patient samples). In some embodiments, the methods provided herein utilize advanced polymer surface coatings, a dual-probe target capture motif with multi-labeled sense oligonucleotides, capture probes comprising locked nucleotides, and/or highly sensitive detectable molecules (e.g., quantum dots).

Methods of the disclosure can, in some embodiments, allow for the determination of a concentration of a target nucleic acid (e.g., a nucleic acid that is representative of a pathogen) in a sample (e.g., a sample from a patient, e.g., having or suspected of having a pathogenic infection). In some embodiments, a method of the disclosure does not require determination of a nucleic acid concentration and instead functions to determine whether a particular target nucleic acid is present within a sample.

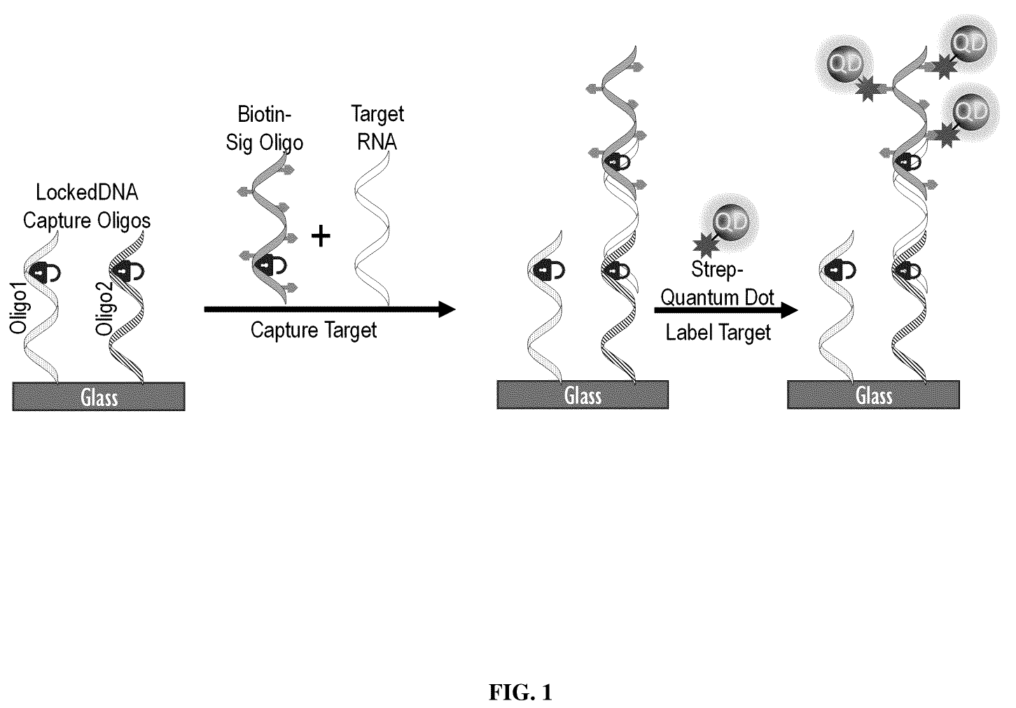

A method can, in some embodiments, involve contacting a sample comprising a target nucleic acid with a sense oligonucleotide (functionalized with a first member of a binding pair) and a capture oligonucleotide that is immobilized to a surface via a silane-containing perfluorinated linker to produce a surface-immobilized target nucleic acid. The target nucleic acid comprises a first region that is complementary to (e.g., and binds to) a capture domain of a capture oligonucleotide and a second region that is complementary to (e.g., and binds to) a target-binding domain of a sense oligonucleotide. In some embodiments, the first region of the target nucleic acid does not overlap with the second region of the target nucleic acid (e.g., the first region and the second region do not share any common nucleotides). A pair of nucleic acid segments (e.g., the first region of a target nucleic acid and a capture domain of a capture oligonucleotide) are “complementary to” one another if the first nucleic acid segment of the pair contains nucleotides that base pair (hybridize/bind through Watson-Crick nucleotide base pairing) with nucleotides of the second nucleic acid segment of the pair such that the two nucleic acid segments form a paired (double-stranded) or partially-paired molecular species/structure. Complementary nucleic acid segments do not need to be perfectly (100%) complementary to form a paired structure, although perfect complementarity is provided, in some embodiments. Addition of the sense oligonucleotide and the target nucleic acid to the surface-immobilized capture oligonucleotide produces a structure comprising the target nucleic acid bound to the capture oligonucleotide and the sense oligonucleotide. A detectable molecule functionalized with a second member of a binding pair can then be added to the structure comprising the target nucleic acid bound to the capture oligonucleotide and the sense oligonucleotide. The presence of the second member of the binding pair allows for the detectable molecule to become associated with (e.g., bound to) the sense oligonucleotide, thereby enabling the detection of the target nucleic acid. A schematic of an exemplary method is provided in FIG. 1.

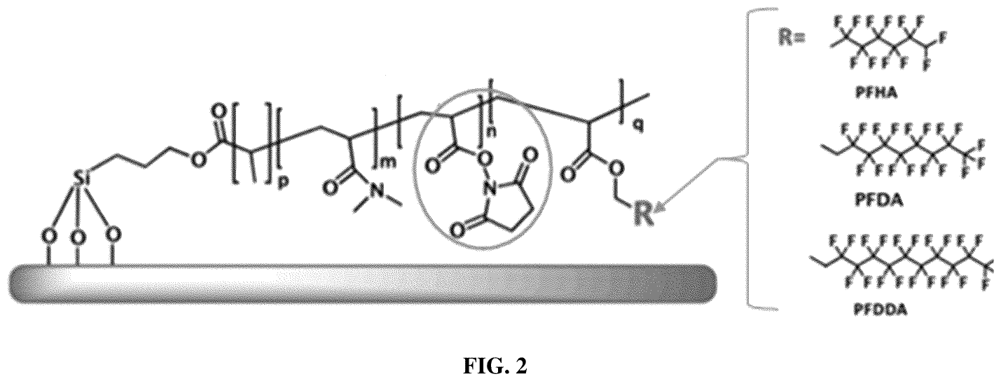

A capture oligonucleotide comprises a capture domain that is complementary to a first region of the target nucleic acid. The capture oligonucleotide is, in some embodiments, immobilized to a surface (e.g., a glass surface, a silicon surface, a silicon oxide surface, or a silicon nitride surface), a polymer surface (e.g., a polypropylene surface, a polystyrene surface, or a plastic surface), or a metallic surface. The surface form-factor may be a flat surface such as a slide, a solid flat surface, a surface comprising multiwells, a bead such as a solid-phase bead (e.g., a magnetic bead, a glass bead), particles such as glass particles microparticles or polymer particles, a microfluidic channel, a nanoaperture, a resin, a matrix, or a membrane. In some embodiments, the capture oligonucleotide is immobilized to the surface with a unique polymer coating. In some embodiments, this polymer coating is a commercial product from Lucidant Polymers known as MCP-2F. In some embodiments, a polymer coating is composed of a co-polymer containing with silane end functionality that anchors the polymer end-on to the glass substrate through silane condensation chemistry. Such a polymer coating can be a silane-containing perfluorinated linker. In some embodiments, a silane-containing perfluorinated linker comprises acrylamide monomers (e.g., for aqueous solubility), NHS ester monomers (e.g., for subsequent carbodiimide coupling of amine functional oligonucleotides such as capture oligonucleotides), and perfluorinated side groups (e.g., to reduce non-specific binding of molecules at the surface). In some embodiments, a silane-containing perfluorinated linker is as provided in FIG. 2 and/or Formula (I):

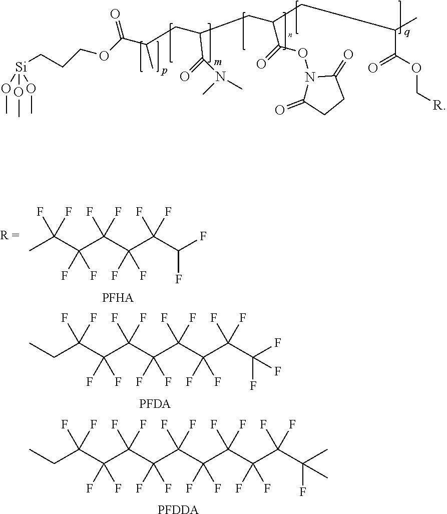

A sense oligonucleotide is functionalized with a first member of a binding pair and comprises a target-binding domain that is complementary to a second region of the target nucleic acid. In some embodiments, the first member of a binding pair is a biotin molecule, an antibody, aptamer, antigen, protein receptor, peptide that binds a protein receptor, or protein receptor.

In some embodiments, a sense oligonucleotide can be functionalized with biotin. In some embodiments, a sense oligonucleotide is functionalized with biotin by reacting a locked nucleotide with Mirus reagent. In other embodiments, a sense oligonucleotide is functionalized with biotin by generating a multi-biotin functionalized oligonucleotide through direct design.

In some embodiments, a capture oligonucleotide (or a target-binding domain of a capture oligonucleotide) and/or a sense oligonucleotide (or a target-binding domain of a sense oligonucleotide) comprises one or more locked nucleotides (LNAs).

The number and positions of the LNAs for a given capture or sense oligonucleotide can be determined using an optimization function which considers all possible combinations of locked positions and computes the Tm for each combination. Combinations which yield the highest Tms are of greatest interest. One of ordinary skill in the art will be capable of using optimization functions, which are known in the art, to design suitable oligonucleotides for use in the methods provided herein. The capture and sense oligonucleotides provided herein were designed in that manner. Some include 6 LNAs at positions that determined by the optimization function.

In some embodiments, a sense oligonucleotide comprises or consists of 1, 2, 3, 4, 5, 6, 7, 8, 9, or 10 LNA, including 1 to 10 LNA, 1 to 9 LNA, 1 to 8 LNA, 1 to 7 LNA, 1 to 6 LNA, or 1 to 5 LNA. In some embodiments, the sense oligonucleotide comprises 5 or 6 LNA. In some embodiments, the capture oligonucleotide comprises 5 or 6 LNA. In some embodiments, the sense and the capture oligonucleotides each comprise 5 or 6 LNA. In some embodiments, the sense oligonucleotide is about 15-20 nucleotides long and comprises 5 or 6 LNA. In some embodiments, the capture oligonucleotide is about 40 nucleotides long and comprises 5 or 6 LNA.

In some embodiments, a capture and/or a sense oligonucleotide comprises or consists of 5-200, 5-150, 5-100, 5-50, 5-25, 10-200, 10-100, 10-50, 25-200, 25-100, 25-50, 50-200, 50-100, or 100-200 nucleotides in length. In some embodiments, the capture oligonucleotide is about 50 nucleotides in length or less (e.g., about 40 nucleotides). In some embodiments, the sense oligonucleotide is about 10 nucleotides in length or less (e.g., about 17-18 nucleotides). The target binding domain of the capture and/or sense oligonucleotide may represent at least 50%, at least 60%, at least 70%, at least 80%, at least 90% or more of the length of the capture and/or sense oligonucleotide, respectively.

A target nucleic acid can be a deoxyribonucleic acid (DNA) or a ribonucleic acid (RNA). In some embodiments, a target nucleic acid comprises a mixture of DNA and RNA nucleotides. In some embodiments, a target nucleic acid is single-stranded (e.g., single-stranded RNA). In some embodiments, a target nucleic acid is double-stranded (e.g., double-stranded DNA).

In some embodiments, a target nucleic acid is a naturally occurring nucleic acid. In some embodiments, a target nucleic acid is a synthetic nucleic acid. In some embodiments, a target nucleic acid is derived from or obtained from a biological sample. A target nucleic acid can be an antigen (e.g., an antigen belonging to a pathogen). A target nucleic acid can encode an antigen (e.g., an antigen belonging to a pathogen) in whole or in part. A biological sample can be a single cell, mammalian cell tissue, animal sample, fungal sample, or plant sample. In some embodiments, a biological sample is a blood sample, saliva sample, sputum sample, fecal sample, urine sample, buccal swab sample or other swab sample, amniotic sample, seminal sample, synovial sample, spinal sample, or pleural fluid sample. In some embodiments, a biological sample is from a human, a non-human primate, a rodent, a dog, a cat, a horse, or any other mammal. In some embodiments, a sample is a purified sample of nucleic acids that have been previously extracted via user-developed methods from metagenomic samples or environmental samples. In some embodiments, a sample is a purified sample of cells or phages that is manipulated to extract DNA or RNA. In some embodiments, a sample comprises genomic RNA and/or genomic DNA.

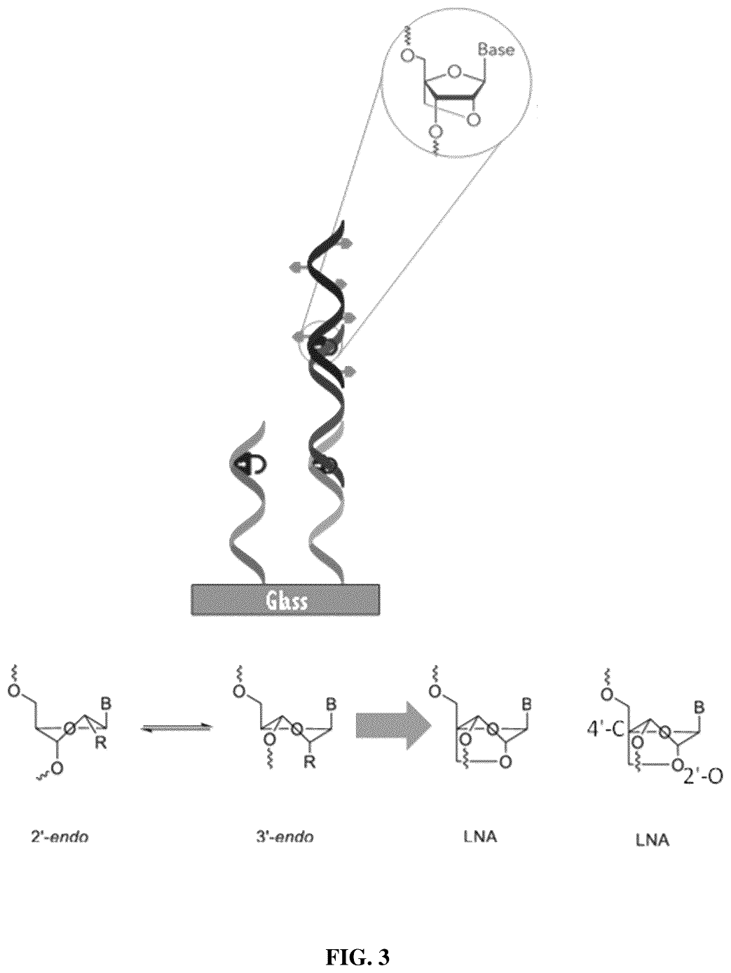

As described, a capture domain and/or a target-binding domain comprises one or more locked nucleotides (LNAs). A locked nucleotide is a modified RNA nucleotide in which the ribose moiety is modified with an extra bridge connecting the 2T oxygen and 4′ carbon. In some embodiments, the ribose moiety is modified such that the 2′ oxygen is bridged to the 4′ carbon by a methylene group. This 2′-O to 4′-C link locks the ribose ring in its 3′-endo conformation (e.g., as seen in FIG. 3). This restricted conformation places the entire base into the most ideal structure for Watson-Crick binding between base pairs as opposed to standard DNA where the ribose ring can transition between adopting 3′-endo and 2′-endo conformations. In some embodiments, locked nucleotides impart a 2-8° C. increase in melting point per locked nucleotide for hybridized base pairs, which can, in some embodiments, result in increased rates and extent of target nucleic acid detection and capture.

Exemplary capture and sense oligonucleotide combinations specific for the nucleic acid targets described herein are as follows:

### BH Capture Oligonucleotide:

/5AmMC6/TA+CCG+CGT G+TA CAT AGT A+CA CA+C CCG CA+G CT+T/3ddC/, wherein a nucleotide that is preceded by “+” is a locked nucleotide, /5AmMC6/ refers to a 5′ amine spaced by 6 methylenes, and /3ddC/ refers to 3′dideoxy cytosine (SEQ ID NO:1);

### BH Sense Oligonucleotide:

/52-Bio/GG TT+T AT+C G+C/iBiodT/ +TA+G C+CC/3BioTEG/, wherein a nucleotide that is preceded by “+” is a locked nucleotide, /52-Bio/ refers to 2 biotins linked to 5′ by amides, /iBiodT/ refers to an internal biotin on thymine, and /3BioTEG/ refers to a 3′ biotin spaced by triethylene glycol (SEQ ID NO:2);

### 18S Capture Oligonucleotide:

/5AmMC6/TC+CTG T+CC GTG TC+C GGG+CCG GG+T GAG GT+T T+C/3ddC/, wherein a nucleotide that is preceded by “+” is a locked nucleotide, 5AmMC6/ refers to a 5′ amine spaced by 6 methylenes, and /3ddC/ refers to 3′dideoxy cytosine (SEQ ID NO:3);

### 18S Sense Oligonucleotide:

/52-Bio/CG+T+G+T+T+GA+G/iBiodT/+C AAA+TTA A/3BioTEG/, wherein a nucleotide that is preceded by “+” is a locked nucleotide, /52-Bio/ refers to 2 biotins linked to 5′ by amides, /iBiodT/ refers to an internal biotin on thymine, and /3BioTEG/ refers to a 3′ biotin spaced by triethylene glycol (SEQ ID NO:4);

### Flu H1N1, M Gene Capture Oligonucleotide:

/5AmMC6/CAGGTAGATRTTGAAAGATGAGVCTTCTAACCGAGGTCGA, 5AmMC6/ refers to a 5′ amine spaced by 6 methylenes, R refers to A or G, and Y refers to C or T/U (SEQ ID NO:5);

### LP Capture Oligonucleotide:

TT+C TTC C+CC AAA+TCG G+CA+CCA ATG+CTA T TTTTTTTTTTTT/3AmMC6T/, wherein a nucleotide that is preceded by “+” is a locked nucleotide, and 3AmMC6/ refers to a 3′ amine spaced by 6 methylenes (SEQ ID NO:6);

### LP Sense Oligonucleotide:

/52-Bio/CT+TGC ATG+C+C/iFluorT/T+T+A G+CC A, wherein a nucleotide that is preceded by “+” is a locked nucleotide, /52-Bio/ refers to 2 biotins linked to 5′ by amides, and/iFluorT/ refers to fluorescein attached to position 5 of the thymine ring by a 6-carbon spacer arm (SEQ ID NO:7);

### MS2 Capture Oligonucleotide:

/5AmMC6/AG G+TT A+CT T+TG TA+A GCC TG+T GAA+CG, wherein a nucleotide that is preceded by “+” is a locked nucleotide, and 5AmMC6/ refers to a 5′ amine spaced by 6 methylenes (SEQ ID NO: 8);

### MS2 Sense Oligonucleotide:

/52-Bio/GC+GCA+GAG+C/iFluorT/C+TGA+CG+A A, wherein a nucleotide that is preceded by “+” is a locked nucleotide, /52-Bio/ refers to 2 biotins linked to 5′ by amides, and /iFluorT/ refers to fluorescein attached to position 5 of the thymine ring by a 6-carbon spacer arm (SEQ ID NO: 9).

A detectable molecule, as provided herein, is a molecule that emits a detectable signal (e.g. a fluorescent or chemiluminescent signal). In some embodiments, a detectable molecule is a fluorophore. In some embodiments, a detectable molecule is a quantum dot. In some embodiments, a detectable molecule is a fluorescent protein or an enzyme.

Examples of fluorophores that can be used herein include, without limitation, hydroxycoumarin, methoxycoumarin, Alexa fluor, aminocoumarin, Cy2, FAM, Alexa fluor 405, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5 581, Rox, Alexa fluor 568, Red 613, Texas Red, Alexa fluor 594, Alexa fluor 633, Allophycocyanin, Alexa fluor 647, Cy5, Alexa fluor 660, Cy5.5, TruRed, Alexa fluor 680, Cy7 and Cy7.5.

A quantum dot, also referred to as a semiconductor nanocrystal, is a small semiconductor crystal having a size of 1-10 nm that emits a fluorescent signal. In some embodiments, a quantum dot comprises elements such as Ag, Cd, Cu, Ga, Hg, In, Ln, P, Pb, S, Se, Si, Te, and Zn. A quantum dot having a size of 5-6 nm in diameter emits longer wavelengths, with colors such as orange, or red. A quantum dot having a size of 2-3 nm emits shorter wavelengths, with colors such as blue and green. In some embodiments, a quantum dot comprises an internal semiconductor core of CdSe coated with an outer shell of ZnS. The ZnS shell is accountable for the chemical and optical stability of the CdSe core in such embodiments. In some embodiments, a quantum dot is an efficient emitter of fluorescent signal and is resistant to quenching upon prolonged excitation. In some embodiments, a quantum dot binding step is facilitated by incubation in a non-protein blocker (e.g., to reduce background signal from non-specific binding of the quantum dot(s) to the surface).

A given quantum dot may have a broad excitation spectrum while exhibiting a sharp emission peak. For this reason, the quantum dots used in the working examples are excited at 488 nm in some experiments and at 635 nm in others. Regardless, the particular quantum dot used emits at 655 nm.

A detectable molecule is, in some embodiments, functionalized with (or conjugated to) a second member of a binding pair. In some embodiments, a second member of a binding pair is an avidin molecule, an antibody, aptamer, antigen, protein receptor, peptide that binds a protein receptor, or protein receptor. Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof.

In some embodiments, one member of a binding pair is a biotin molecule (or biotin, as those terms are used interchangeably herein) and the other member of the binding pair is an avidin protein (e.g., streptavidin). In some embodiments, one member of a binding pair is an antibody or aptamer and other member of the binding pair is an antigen that binds to the antibody or aptamer. In some embodiments, one member of a binding pair is a first protein and other member of the binding pair is a second protein that binds to the first protein. In some embodiments, one member of a binding pair is a protein receptor and other member of the binding pair is a peptide that binds to the protein receptor.

In the working examples, a quantum dot conjugated to streptavidin having an emission maxima of about 655 nm (e.g., Qdot™ 655 Streptavidin Conjugate, commercially available from Invitrogen) was used to detect all nucleic acid targets. The quantum dot may comprise a plurality of binding partners, such as streptavidin. In some embodiments, there are 5-10 binding partners (e.g., streptavidin) per quantum dot nanocrystal.

The assay is typically carried out in the presence of a buffer. Exemplary buffers may comprise about 4×SSC to about 6×SSC and about 0.05% to about 2.0% Tween (e.g., Tween-20). One example of a suitable buffer is a 5×SSC/0.1% Tween (e.g., Tween-20) buffer.

Other factors may be modulated in order to maximize signal from the assay. As demonstrated in the Examples, the conditions during printing of capture oligonucleotides onto slides may be varied, including relative humidity.

A method described herein can be performed at any reasonable temperature. For example, any step of the method can be performed at a temperature of 4-40° C., 4-37° C., 4-30° C., 4-25° C., 4-15° C., 4-10° C., 10-40° C., 15-37° C., 15-25° C., or room temperature. Similarly, any step of the method can be performed for any reasonable period of time. For example, any step of the method can be performed for 5-60 minutes, 5-300 minutes, 5-200 minutes, 5-100 minutes, 30-180 minutes, 1-4 hours, 1-3 hours, or 1-2 hours. A method can be performed in a reaction mixture (e.g., in a reaction vessel). A reaction mixture can be a liquid that includes a buffer, water, metal ions, etc.

Some methods involve removing nucleic acids that are not bound to the capture oligonucleotide from a reaction mixture. Removal of unbound nucleic acids from a reaction mixture can be performed by washing the reaction mixture (e.g., with a wash buffer). A wash buffer can be a high-salt wash buffer, low-salt wash buffer, or phosphate-buffered saline.

The methods described herein may be performed manually or using automated devices. Such automated devices may also be capable of processing a cell or phage sample to extract target nucleic acids, and optionally may convert a target nucleic acid into another form (e.g., the device may convert an RNA into a cDNA). The methods do not require that RNA be converted to cDNA.

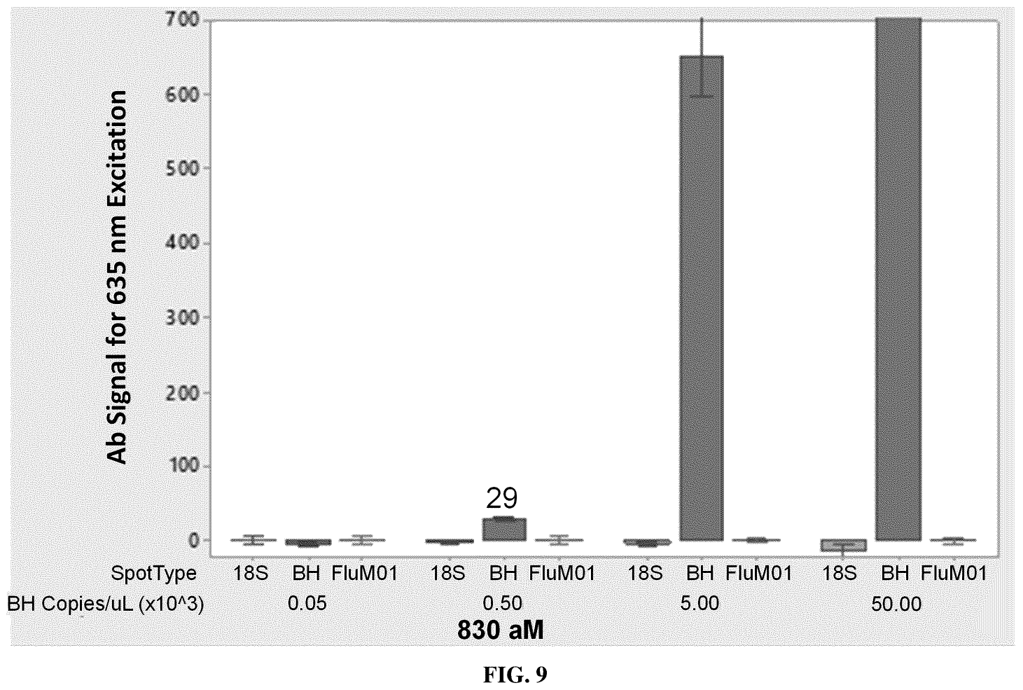

The methods described herein were demonstrated to achieve a limit of detection of about 500 copies of a target nucleic acid per microliter (i.e., on the order of about 830 attomolar (aM), as shown in FIG. 9.

The methods may also be used in multiplexed assays to detect more than one target at a time. As demonstrated in the Examples, various two or three target combinations may be detected simultaneously, with little crosstalk. As an example, the methods were able to detect RNA for the human marker 18S and a bacterial BH marker simultaneously, and 18S and MS2 RNA simultaneously, and BH and MS2 RNA simultaneously, and 18S, BH and MS2 RNA simultaneously. MS2 is a non-infectious viral simulant that is useful for evaluating diagnostic capabilities.

Unlike other detection methodologies, the methods provided herein do not require amplification of the sample (i.e., of the target nucleic acid). This avoids, among other things, errors that may be introduced during amplification (resulting in loss of signal for a target) or bias that may occur during amplification (resulting in a representation profile that is different from the original sample, as well as loss of target altogether). The level of sensitivity and specificity of the method when using an automated device is on par with currently available detection systems such as BioFire and QiaStat. This is true for both bacterial and viral target detection.

Further, when using an automated system to perform the method, in whole or in part, the time to completion can be relatively short, on the order of 1-2 hours.

### EXAMPLES

### Example 1. Development of an Exemplary Method of the Disclosure

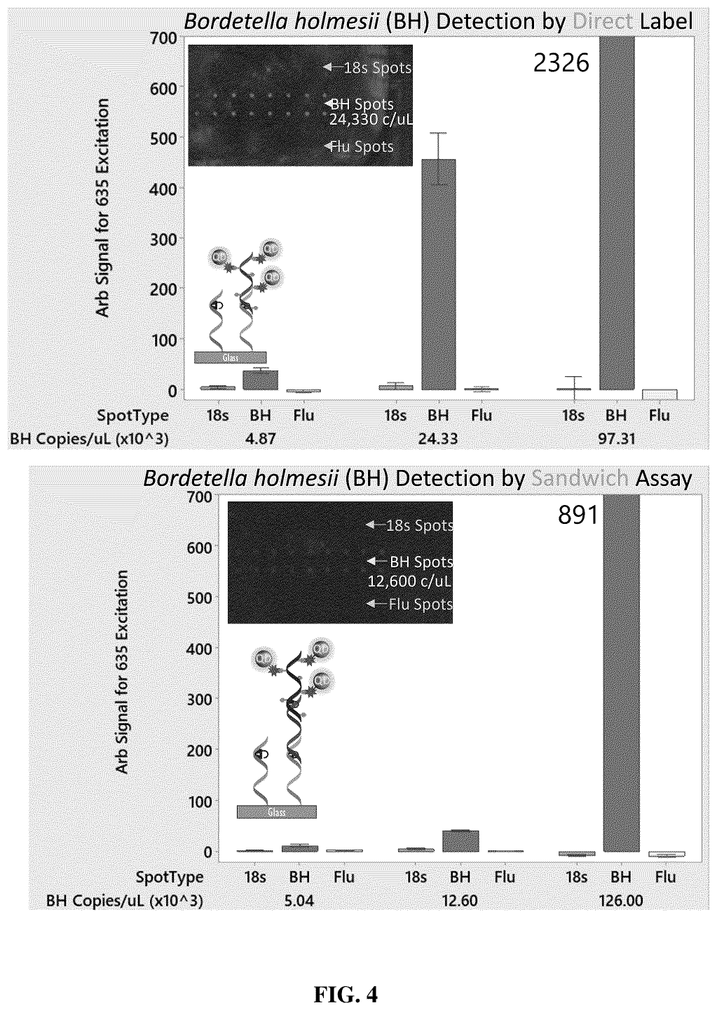

Initial experiments utilizing a method of the disclosure showed great promise as displayed in FIG. 4. Experiments were set up to evaluate the detection of genomic RNA from the bacterial pathogen Bordetella Holmesii (BH) at different loadings as measured in copies per microliter (copies/μL). It was found that target RNA could be observed when directly labeled with biotin (by Mirus reaction) or when sandwiched between two oligonucleotides (a sense oligonucleotide functionalized with biotin and a surface-immobilized capture oligonucleotide). Most importantly, signal, while low, was observed for concentrations in the range of 5×10³ copies/μL (4.87 and 5.05×10³ in Figures) which is within a clinically relevant range.

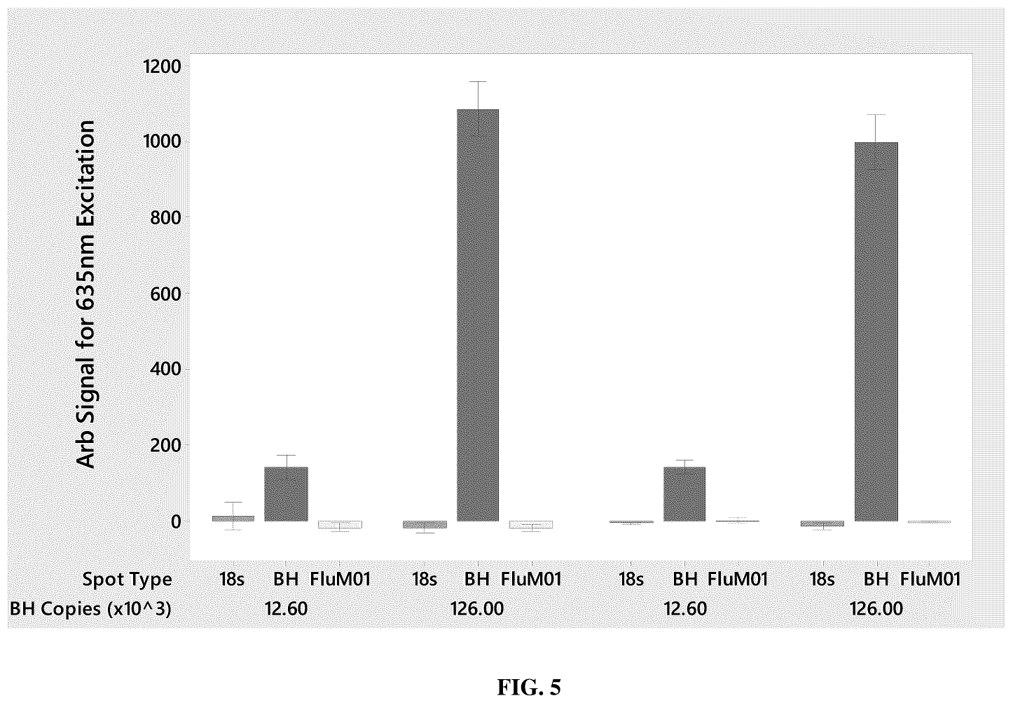

These experiments were replicated using only the sandwich assay (i.e., an exemplary method of the disclosure), omitting the directly labeled target variation. The process was refined by making multiple slide coatings and prints. As a result, the detection of genomic BH RNA was confirmed and signal was improved by about 3× over an initial trial as shown in FIG. 5 which represented slide preparation conditions having 70% (first 6 bars) or 40% (last 6 bars) relative humidity (RH).

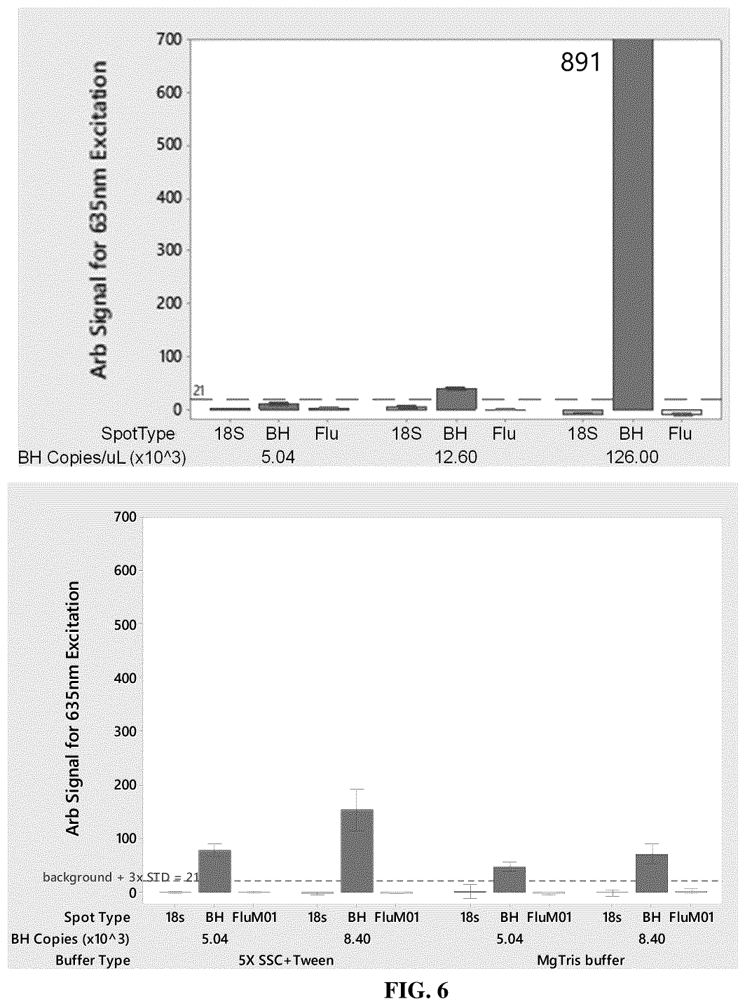

Signal was then further improved through optimization of the assay buffer. It was determined that a buffer with 5×SSC and 0.1% Tween-20 provided a significant increase in signal, on the order of 7× over the initial result, as shown in FIG. 6. The new buffer provided signal at 5×10³ copies/μL that was well above baseline+3×STD (i.e., typically a rough threshold used to establish that an assay signal is sufficient to be diagnostic).

### Example 2. Further Method Development

The next stage of development involved implementing custom designed sense oligonucleotides with multiple biotins pre-installed. These oligos were designed to have two biotins at the 5′ end, one internal biotin and one biotin at the 3′end.

The specific structure of the BH capture oligonucleotide was /5AmMC6/TA+CCG+CGT G+TA CAT AGT A+CA CA+C CCG CA+G CT+T/3ddC/, wherein a nucleotide that is preceded by “+” is a locked nucleotide, /5AmMC6/ refers to a 5′ amine spaced by 6 methylenes, and /3ddC/ refers to 3′dideoxy cytosine (SEQ ID NO:1).

The specific structure for the BH sense oligonucleotide was /52-Bio/GG TT+T AT+C G+C/iBiodT/ +TA+G C+CC/3BioTEG/, wherein a nucleotide that is preceded by “+” is a locked nucleotide, /52-Bio/ refers to 2 biotins linked to 5′ by amides, /iBiodT/ refers to an internal biotin on thymine, and /3BioTEG/ refers to a 3′ biotin spaced by triethylene glycol (SEQ ID NO:2).

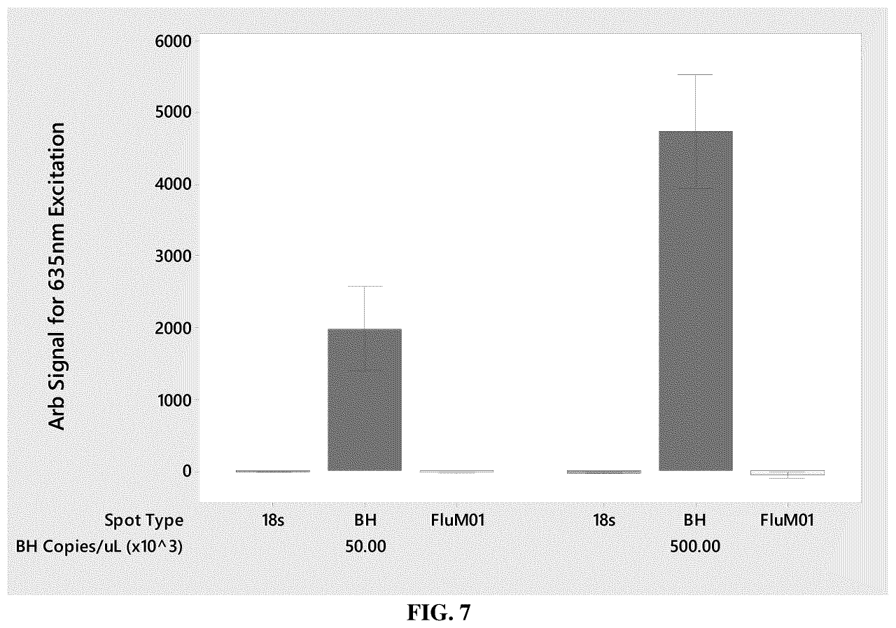

Assays run on genomic BH RNA with biotinylated sense oligonucleotides showed a drastic improvement in signal yielding approximately an 11× increase over the initial result, as shown in FIG. 7.

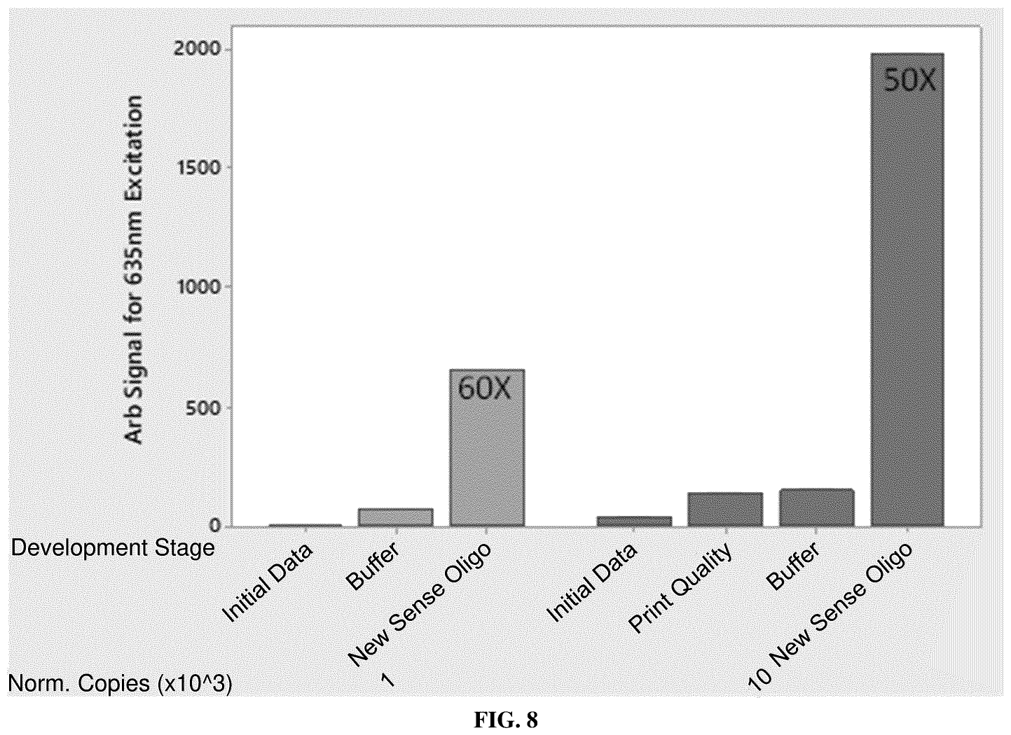

Overall, the optimization process relating to oligonucleotide printing conditions and buffer selection, and implementation of biotinylated sense oligonucleotides improved signal for genomic BH RNA by 60× at 1×10³ copies/μL and about 50× at 1×10⁴ copies/μL level, as shown in FIG. 8.

Leveraging all improvements in signal strength increased the assay sensitivity enough to enable detection of genomic BH RNA down to concentrations of 500 copies/μL which is within the attomolar range, as shown in FIG. 9.

To verify the agnostic nature of the chemistry, the assay signal was evaluated for a second RNA target, 18S, which is a human biomarker.

The specific structure of the 18S capture oligonucleotide was /5AmMC6/TC+CTG T+CC GTG TC+C GGG+CCG GG+T GAG GT+T T+C/3ddC/, wherein a nucleotide that is preceded by “+” is a locked nucleotide, 5AmMC6/ refers to a 5′ amine spaced by 6 methylenes, and /3ddC/ refers to 3′dideoxy cytosine (SEQ ID NO:3).

The specific structure of the 18S sense oligonucleotide was /52-Bio/CG+T+G+T+T+GA+G/iBiodT/+C AAA+TTA A/3BioTEG/, wherein a nucleotide that is preceded by “+” is a locked nucleotide, /52-Bio/ refers to 2 biotins linked to 5′ by amides, /iBiodT/ refers to an internal biotin on thymine, and /3BioTEG/ refers to a 3′ biotin spaced by triethylene glycol (SEQ ID NO:4).

The specific structure of the Flu H1N1 M Gene capture oligonucleotide was /5AmMC6/CAGGTAGATRTTGAAAGATGAGYCTTCTAACCGAGGTCGA, 5AmMC6/ refers to a 5′ amine spaced by 6 methylenes, R refers to A or G, and Y refers to C or T/U (SEQ ID NO:5).

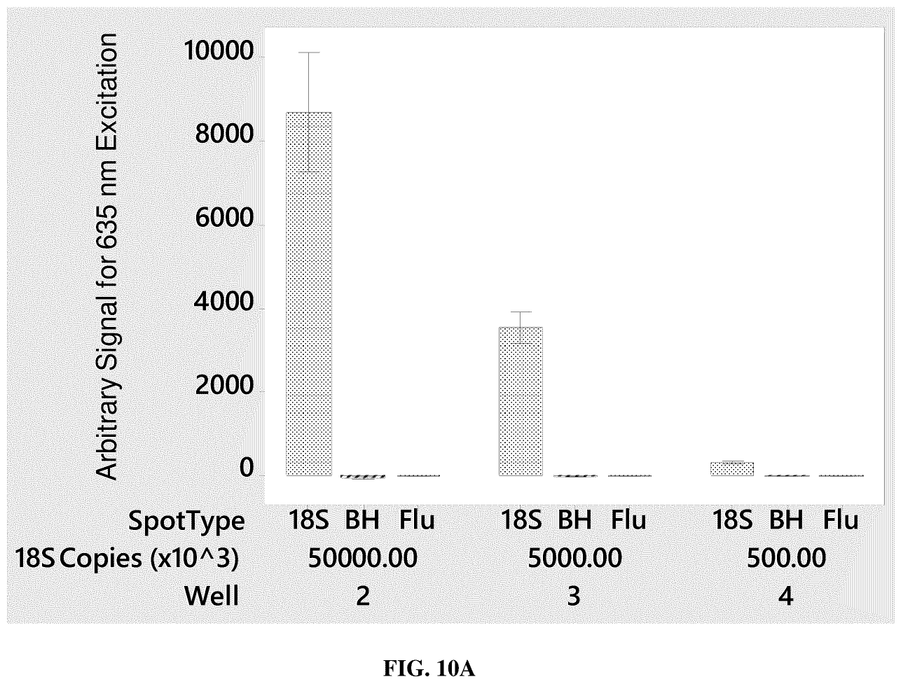

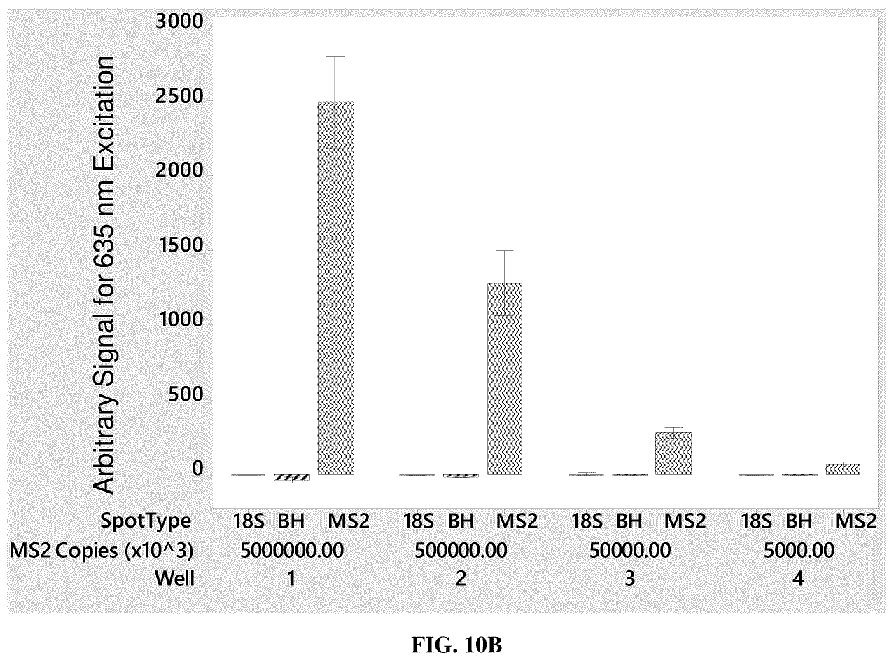

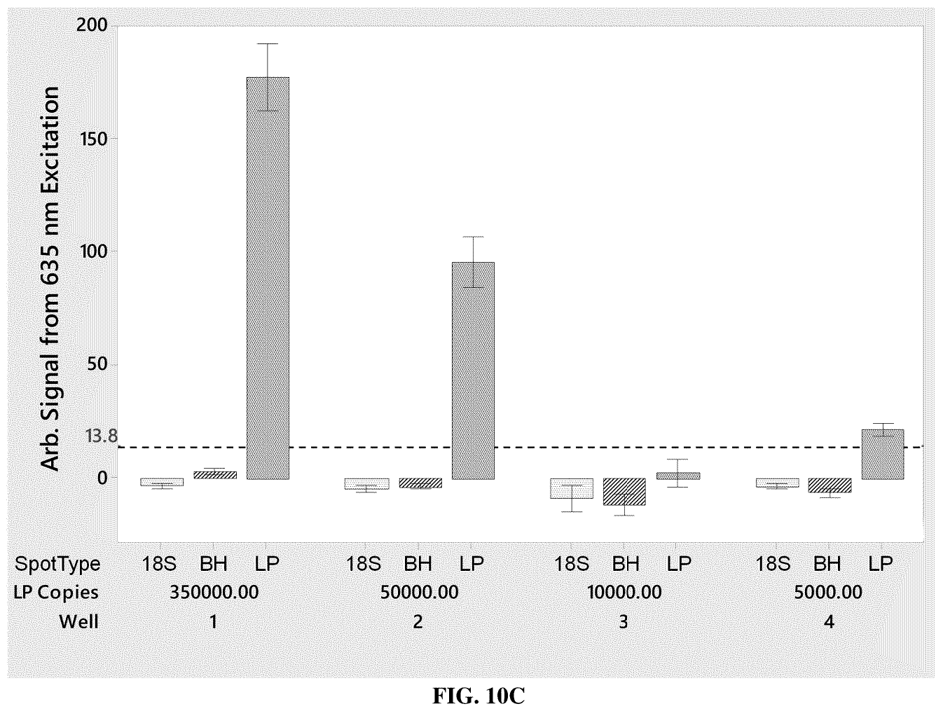

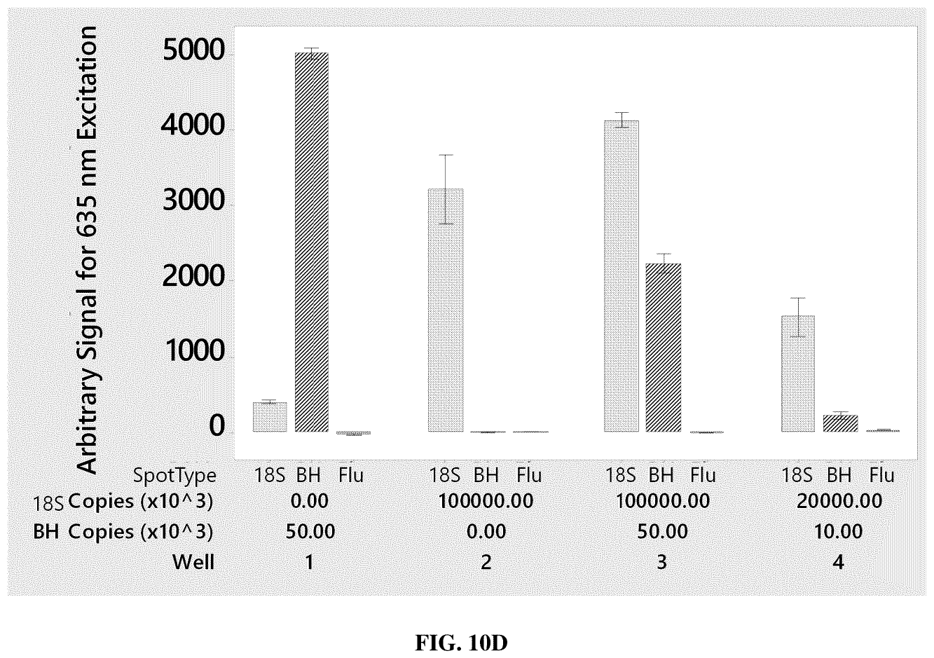

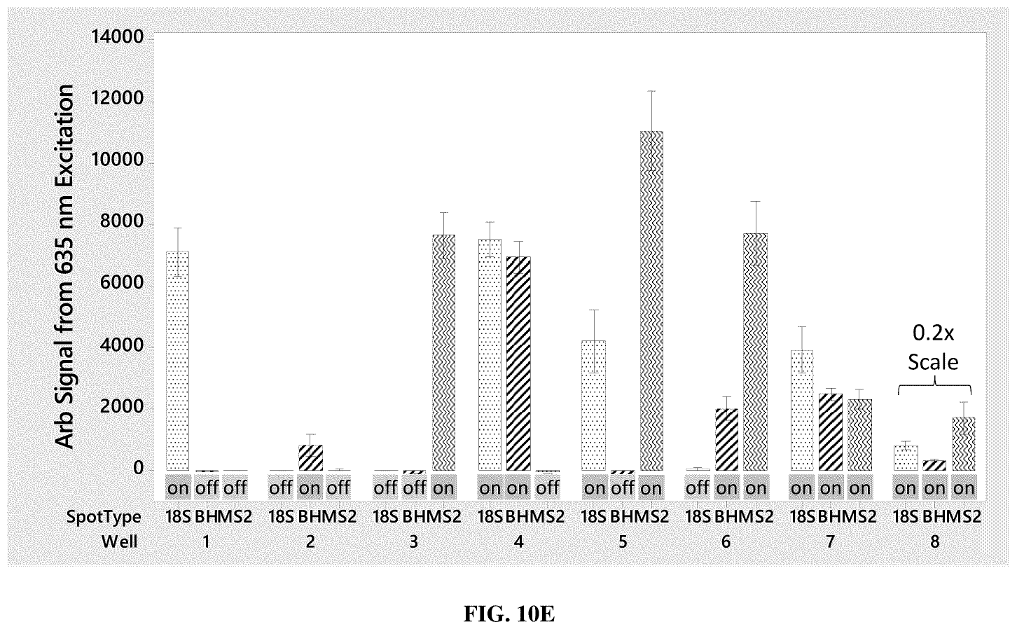

Assay signal for 18S was found to be strong and highly specific as depicted in FIG. 10A, supporting the ability of this chemistry to cleanly detect an additional target.

The assay was also used to detect target RNA from virus MS2 (FIG. 10B) and from bacteria Legionella pneumophila (LP) (FIG. 10C).

The specific structure of the LP capture oligonucleotide was TT+C TTC C+CC AAA+TCG G+CA+CCA ATG+CTA T TTTTTTTTTTTT/3AmMC6T/, wherein a nucleotide that is preceded by “+” is a locked nucleotide, and 3AmMC6/ refers to a 3′ amine spaced by 6 methylenes (SEQ ID NO:6).

The specific structure of the LP sense oligonucleotide was /52-Bio/CT+TGC ATG+C+C/iFluorT/T+T+A G+CC A, wherein a nucleotide that is preceded by “+” is a locked nucleotide, /52-Bio/ refers to 2 biotins linked to 5′ by amides, and/iFluorT/ refers to fluorescein attached to position 5 of the thymine ring by a 6-carbon spacer arm (SEQ ID NO:7).

The specific structure of the MS2 capture oligonucleotide was /5AmMC6/AG G+TT A+CT T+TG TA+A GCC TG+T GAA+CG, wherein a nucleotide that is preceded by “+” is a locked nucleotide, and 5AmMC6/ refers to a 5′ amine spaced by 6 methylenes (SEQ ID NO: 8).

The specific structure of the MS2 sense oligonucleotide was /52-Bio/GC+GCA+GAG+C/iFluorT/C+TGA+CG+A A, wherein a nucleotide that is preceded by “+” is a locked nucleotide, /52-Bio/ refers to 2 biotins linked to 5′ by amides, and /iFluorT/ refers to fluorescein attached to position 5 of the thymine ring by a 6-carbon spacer arm (SEQ ID NO: 9).

### Example 3. Multiplexing Capability

In addition to being sensitive, the method was also shown to be capable of multiplexing (e.g., to detect multiple targets). In a multiplexing assay, capture oligonucleotides are printed onto surfaces in a spatially separate manner. In this way, a single detectable label, such as a quantum dot, may be used to bind to all sense oligonucleotides that are immobilized to the surface (via binding to their respective immobilized target). Signal from different targets may be distinguished based on the location of the signal on the surface. This results in a further simplified detection method, and removes any variability that may occur when using different quantum dots or altogether different detectable labels between different targets.

To test multiplexing, assays were run with BH genomic RNA and 18S genomic RNA. Results, provided in FIG. 10D, showed that signal was specific to the targets present with single wells showing signal for either BH or 18S and duplex wells showing signal for both targets. No crosstalk was observed to the off-target Flu H1N1, M Gene while a small amount signal was observed on the 18S in the BH only well (well 1). This was later addressed by increasing the assay temperature to anneal off non-specific oligonucleotide hybridization. Further results, provided in FIG. 10E, demonstrate the ability to detect 18S and MS2 RNA, BH and MS2 RNA, and 18S, BH and MS2 RNA simultaneously.

### Example 4. Automation Compatibility

The methods of the disclosure can be performed with automated systems to enable detection of RNA and DNA targets. In this regard, the method should be compatible with microfluidics and retain efficacy when executed by a predefined automation script. This capability was tested by loading the assay components (e.g., target nucleic acids, sense oligonucleotides, quantum dots, and wash buffers) into a custom designed microfluidic cartridge. The cartridge was also fitted with a microchamber containing a glass substrate functionalized with MCP-2F polymer (e.g., as shown in Formula (I)) and printed with locked capture oligonucleotides (i.e., capture oligonucleotides comprising locked nucleotides). The assay was loaded into an automated assay platform (e.g., a massively multiplexed device (MMD)). The MMD was comprised of hardware including pumps, sensors, heaters, and mixers, necessary to run the assay in the cartridge as well as optical components to image the microarray during and upon completion of the assay. The entire system was governed by custom software which coordinates hardware, optics and image processing.

Several assays were run in the automated system, and all produced target-specific signal. Using one such automated system, single-plex runs containing 18S RNA as the target were performed. 18S RNA was detected at two concentration levels (data not shown). Multiplexing was also demonstrated in the automated system with both BH RNA and 18S RNA detected simultaneously (data not shown). In this particular automated assay and system, improvements were made to the automated execution that resulted in signal sufficient enough to saturate the photodiode sensor (data not shown). Image processing was employed to clarify that signal was observed at all individual spots for each target (data not shown).

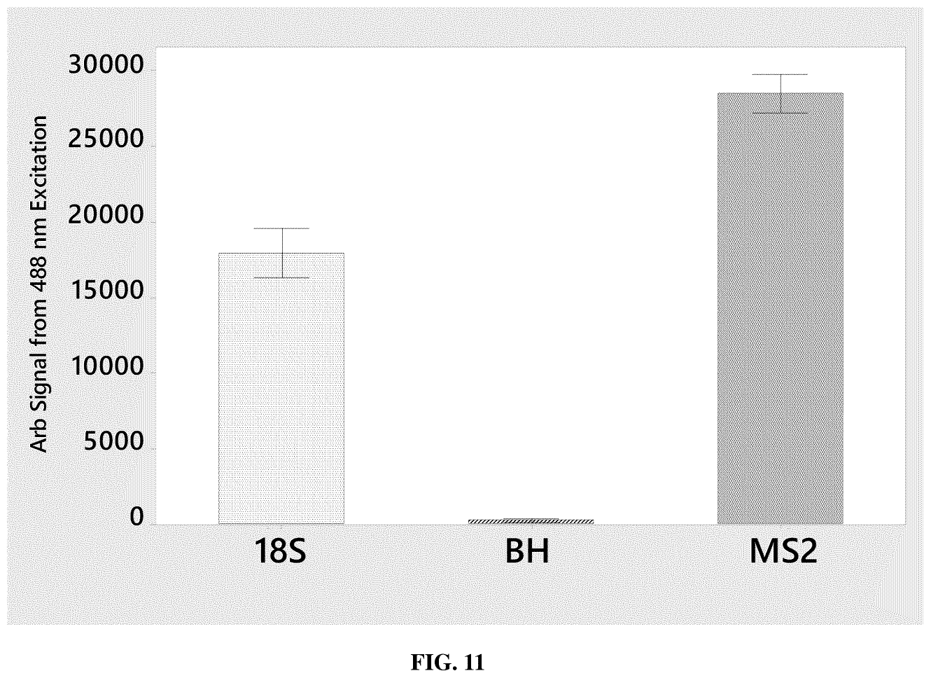

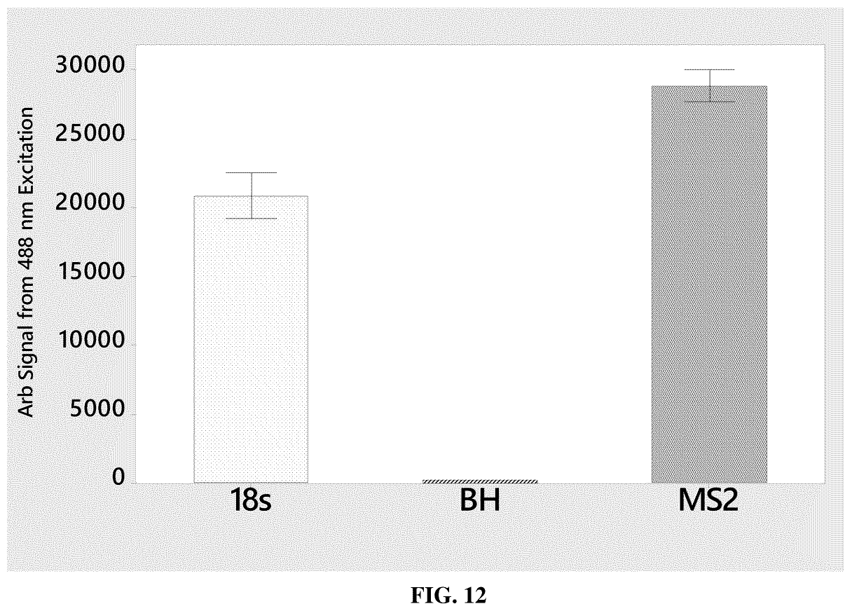

Thereafter, multiplexed detection of MS2 RNA and 18S RNA (provided in the form of AMN) was performed using the automated system. MS2 RNA was provided at a concentration of 5×10⁹ copies per microliter. All targets were passed through the extraction process of the automated system. The total run time was 109 minutes. The results from this experiment are shown in FIG. 11, which shows signal for 18S RNA and MS2 RNA but no signal for BH RNA. Another multiplexed detection of MS2 (in the form of a bacteriophage) and 18S (AMN) was performed using the automated system. This experiment represents a fully automated whole organism viral extraction and detection. MS2 was provided at a concentration of 2×10⁴ PFU per microliter, and although this represented a significant drop in concentration relative to prior experiments, a good signal was still obtained. The total run time was 157 minutes. The results from this experiment are shown in FIG. 12, which shows signal for 18S and MS2 but no signal for BH.

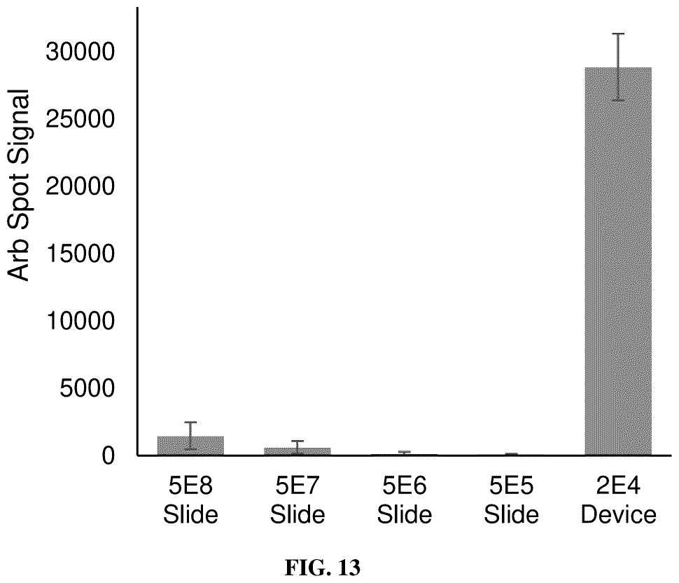

A further experiment was performed to compare the ability of manual and automatic processes to detect low concentration targets. In this experiment, the target was MS2 RNA at varying concentrations. The results are shown in FIG. 13. Using visual detection, MS2 RNA was detected on slides at concentrations of 5×10⁸ and 5×10⁷ copies per microliter. Using automated detection, MS2 was detected at a concentration of 2×10⁴ PFU per microliter. Assuming an average of about 1 RNA copy per PFU, these results suggest that, at low concentrations of target, a much higher signal is achievable using the automated process (in which target nucleic acid is directly extracted from an organism in an automated device) than the manual detection method (in which target nucleic acid is manually extracted from an organism by conventional means and then input into a device). This further suggests the possibility of detecting 2×10³ to 2×10² copies per microliter, without amplification.

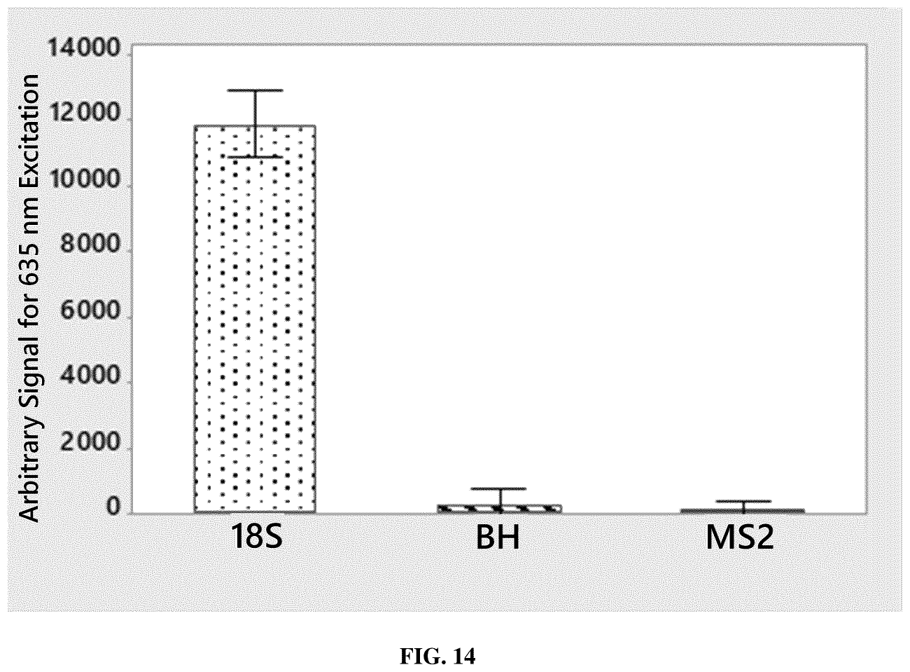

In a final experiment, a swab sample containing 18S (provided in the form of AMN) was analyzed using the automated system. The results are shown in FIG. 14. The total run time was 30 minutes.

In summary, the assay achieved a limit of detection (LOD) as low as 500 copies/μL for one RNA target. This LOD is below the typical pathogen loading in most patient samples. Multiplexing was demonstrated with high specificity for each target detected indicating a lack of interference from multiple sense oligonucleotides and low cross-talk between capture oligonucleotides. The assay was found to be highly compatible with automated systems indicating it can support RNA and DNA detection in point-of-care and point-of-need devices. The methods may be paired with automated systems which provide upstream sample processing, allowing analysis of cell or phage as the starting material (or any other sample containing unextracted DNA or RNA). Further since the method does not require amplification, it is not subject to the various drawbacks or artifacts introduced by amplification techniques.

### Example 5. Experimental Protocols for Examples 1-4

### Glass Slide Surface Coating

Standard 25 mm×17 mm by 1 mm glass slides were washed to clean the substrate and remove any debris. The washing protocol comprised successively immersing the slide in 50 mL conical tubes of the following solutions for the corresponding times:
1) 0.02% Triton-X, 1 min
2) 0.5 mM HCl, 1 min
3) 100 mM KCl, 1 min
4) UltraPure H₂O (UPH₂O), 1 min
5) UltraPure H₂O (UPH₂O), 1 min

After washing the slides were spun dry in 50 mL conical tubes at 500 rcf for 3 min.

Glass slides were activated for the coating by placing in an oxygen plasma for 10 min. After activation, 8 chambered GraceBio Wells were affixed to the glass slides and 240 μL of MCP-2F coating buffer was added to each well. The wells were sealed with foil and the MCP-2F coating buffer was incubated in the wells for 30 min. After incubation, coating solution was aspirated and the GraceBio Wells were removed from the slides. Each slide was washed in 1 L of DIH₂O. Slides were spun dry in 50 mL tubes at 500 rcf for 3 min and baked at 80 C for 30 min.

### Oligonucleotide Printing

Capture oligonucleotides were dissolved in print buffer and printed onto the slides using a Biodot. Capture oligonucleotide print solutions were prepared by diluting 6 μL of 100 mM oligomer in UPH₂O in 24 μL of Lucidant Spot-on print buffer. The oligomer solutions were loaded into the Biodot print tips and printed onto the MCP-2F coated slides in micro-arrays comprising 2×10 spots deposited with 10 drops/spot for each oligo. Printing was done at 70% relative humidity (RH). After printing the slides were incubated in 90% RH overnight. Each slide was then quenched to consume residual NHS-ester functionality by immersing and incubating each slide in 50 mL of 1× Lucidant Block-On solution for 30 min at 50 C. The slides were washed twice with 50 mL UPH₂O for 1 min and spun dry at 500 rcf for 3 min.

### RNA Detection Assay

A slide printed with micro-arrays was affixed with 2×8 GraceBio Wells. Each well was blocked by adding 200 μL of SuperBlock and incubating for 60 min. After incubation the SuperBlock was aspirated from each well and the wells were washed 3 times with 200 μL of 3×SSC containing 1 uL/mL buffer RNase Inhibitor (SUPERase-In RNase Inhibitor). Capture solutions were prepped containing 300 nM SenseOligo and target RNA at designated concentrations (typically 500-10,000 copies/μL), in capture buffer (5×SSC+0.3% Tween-20). 100 μL of capture solution was added to each well and the wells were sealed with foil. The slide was incubated at 80 C for 30 min with orbital agitation at 75 rpm. After target capture, the wells were aspirated and washed 3× with 200 μL of 3×SSC containing 1 uL/mL buffer RNase Inhibitor. Quantum dots were then bound to biotin functionality within the microarrays by adding 100 μL of 1 nM Quantum dots (Qdot 655 streptavidin conjugate) in non-protein blocker (Pierce Protein-Free (TBS) Blocking Buffer) to each well and the wells were sealed with foil. The slide was incubated at 37° C. for 15 min with orbital agitation at 75 rpm. After QD incubation the wells were washed with 1× borate buffer with 0.2% Sodium Dodecyl Sulfate and 1 uL/mL RNAse Inhibitor. At this point, the GraceBio Wells were removed, and the slide was dipped in UPH₂O for 1 second, then spun dry in a 50 mL conical tube for 3 min at 500 rcf.

### Array Imaging

Signal from assays was imaged on an Innopsys InnoScan1100 three-color ultra-high resolution scanner.

### Example 6. Example Processes and Systems

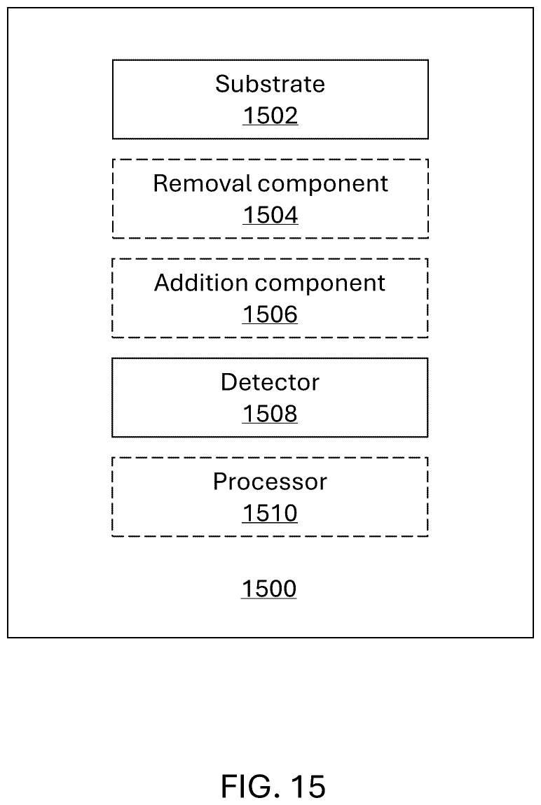

FIG. 15 shows a detection system 1500, according to some embodiments. Detection system 1500 may be a detection system of target nucleic acids. As shown in FIG. 15, detection system 1500 comprises a substrate 1502 and a detector 1508. Detection system 1500 may also optionally comprise a removal component 1504, an addition component 1506, and/or a processor 1510. Additionally or alternatively, information may be transferred between the detection system 1500 and one or more other system that comprise one or more of a removal component 1504, an addition component 1506, and/or a processor 1510.

Substrate 1502 may comprise an aperture (e.g., a cavity or a chamber) or a substrate face configured to receive a surface described above (e.g., a glass surface, such as glass surface on a glass substrate). In some embodiments, substrate 1502 may comprise a surface described above itself. The substrate 1502 may also comprise a combination of the aperture or substrate face and the surface itself. When a surface described above is disposed in the aperture or is arranged at the substrate face, the surface is disposed on the substrate 1502. Fluids or other reaction components may be received by the substrate 1502. For example, a fluid or other reaction component may be added into the aperture or added onto a surface described above (either of which may include a fluid or other reaction component being added onto the surface, and the surface subsequently being received by the aperture).

Detector 1508 may include a sensor, such as an image sensor. For example, the image sensor may comprise a CMOS or CCD image sensor. In some embodiments, the detector 1508 may comprise a camera that includes the image sensor. An image sensor may be configured to capture image data (e.g., one or more image frames) of an image target based on the intensity of light detected at each pixel of a plurality of pixels arranged in an array in the image sensor. Exemplary image data is discussed above. In some embodiments, the detector may further comprise an illumination source (e.g., a visible, infrared, or ultraviolet light source) configured to illuminate an image target (or detectable label such as a quantum dot) while image data is captured by the image sensor. In some embodiments, the detector may comprise an electrical sensor. An electrical sensor may comprise one or more electrodes configured to detect characteristics of a sample based on electrical parameters (e.g., resistance, capacitance, etc.). In some embodiments, the detector may include a sensor, such as one or more of a temperature, pressure, proximity, motion, or position sensor.

The removal component 1504 may be configured to remove elements (such as reaction components, substrates, by-products, unbound components, and the like) from the substrate. For example, the removal component may remove fluids or unreacted reaction components such as unbound sense oligonucleotides or unbound detectable labels (e.g., quantum dots) of a reaction mix from the substrate. The addition component 1506 may be configured to add elements (such as reaction components, substrates, wash solutions, including sense oligonucleotides, buffers, detectable labels such as quantum dots) to the substrate. For example, the addition component may add fluids to a reaction mix of the substrate. In some embodiments, each one of the removal component 1504 or the addition component 1506 may comprise at least one fluid instrument selected from the group consisting of: a channel, an inlet, an outlet, a burette, a nozzle, a valve, a dispenser, a flusher, and a controller configured to control any of the preceding group members. The at least one fluid instrument may be configured to remove and/or add fluids to the substrate. In some embodiments, each one of the removal component 1504 or the addition component 1506 may comprise a container configured to hold a fluid after it is removed and/or before it is added to the substrate 1502.

As described above, the detection system 1500 may optionally comprise the processor 1510. For example, the detection system 1500 may itself include the processor 1510 and/or the detection system 1500 may have its information (e.g., data detected by detector 1508) transferred to another system which comprises a processor 1510 (e.g., via a communication link). It should be appreciated that any functions described herein as being performed by a processor herein may also be performed by one or more processors. For example, a plurality of processors, between the plurality, may perform one or more of the functions described herein. In some embodiment, processor 1510 may control the detection system 1500. For example, the processor 1510 may control the removal component 1504, the addition component 1506, and the detector 1508. The processor 1510 may also control the addition and removal of various elements described herein to the substrate 1502, such as one or more of a sample, a capture oligonucleotide, a sense oligonucleotide, or detectable label such as a quantum dot. Processor 1510 may include some or all of the components of computing system 1700, described below (e.g., storage and memory). In some embodiments the processor may comprise circuitry (e.g., an integrated circuit (IC)) configured to perform the functions described herein, such as an application-specific integrated circuit (ASIC), a microprocessor, or a system-on-chip (SOC).

In some embodiments, the processor 1510 may perform an algorithm described herein. For example, the processor 1510 may perform an algorithm comprising one or more steps of process flow 1600 or 1650, e.g., one or more of step 1602, step 1604, step 1606, step 1608, step 1652, step 1654, or step 1656. In some embodiments, the processor 1510 may cause another component of the detection system 1500 or another system, to perform one or more of step 1602, step 1604, step 1606, step 1608, step 1652, step 1654, or step 1656. In some embodiments, the processor 1510 may perform one or more of these steps in response to a user input.

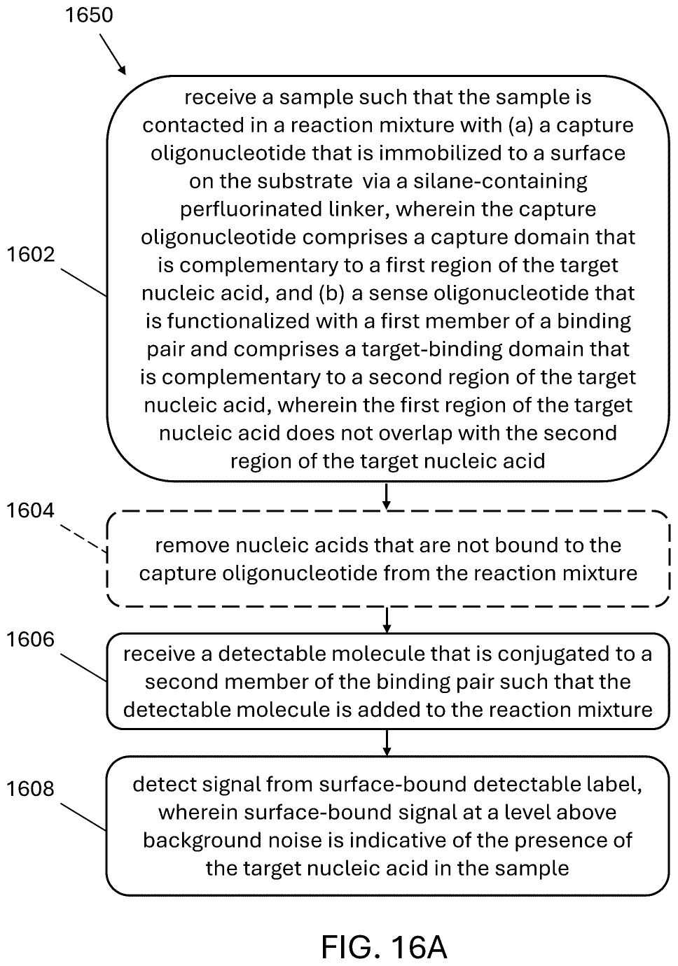

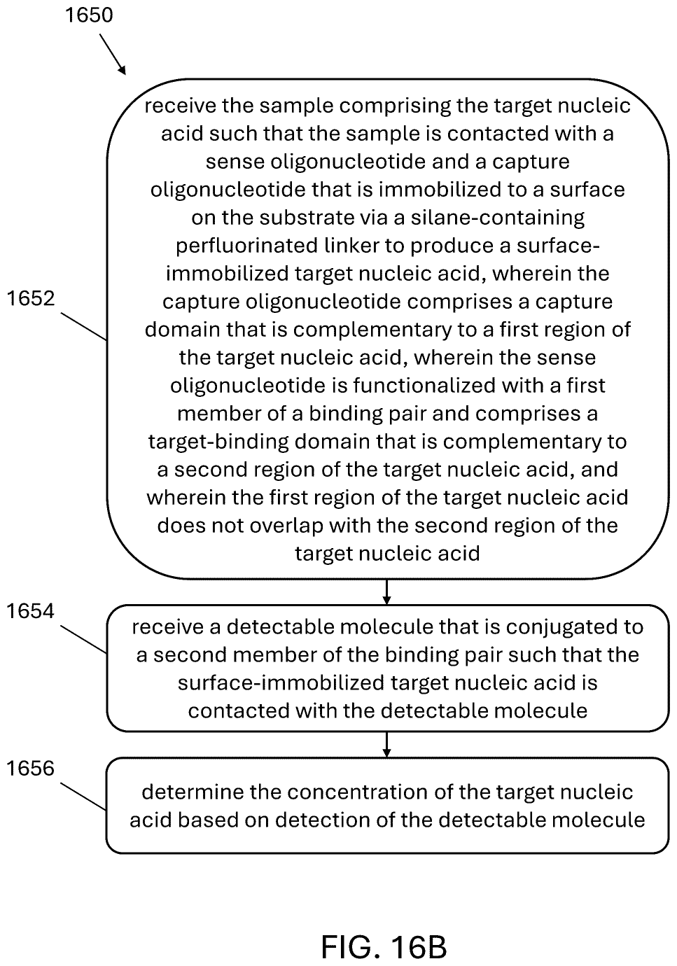

FIG. 16A shows an example process flow 1600 for a method of detecting a target nucleic acid in a sample, according to some embodiments. Process flow 1600 may include step 1602, optional step 1604, step 1606, and step 1608. Detection system 1500 may perform process flow 1600.

At step 1602, the system receives a sample such that the sample is contacted in a reaction mixture with (a) a capture oligonucleotide that is immobilized to a surface on the substrate via a silane-containing perfluorinated linker, wherein the capture oligonucleotide comprises a capture domain that is complementary to a first region of the target nucleic acid, and (b) a sense oligonucleotide that is functionalized with a first member of a binding pair and comprises a target-binding domain that is complementary to a second region of the target nucleic acid, wherein the first region of the target nucleic acid does not overlap with the second region of the target nucleic acid. In some embodiments, the substrate 1502 may perform step 1602. In some embodiments, the addition component 1506 may add one or more of the sample, the capture oligonucleotide, or the sense oligonucleotide to the substrate 1502.

At optional step 1604, the system removes nucleic acids that are not bound to the capture oligonucleotide from the reaction mixture. In some embodiments, the removal component 1504 may perform step 1604.

At step 1606, the system receives a detectable molecule that is conjugated to a second member of the binding pair such that the detectable molecule is added to the reaction mixture. In some embodiments, the substrate 1502 may perform step 1606. In some embodiments, the addition component 1506 may add the detectable molecule to the reaction mixture, at the substrate 1502.

At step 1608, the system detects signal from surface-bound detectable label, wherein surface-bound signal at a level above background noise is indicative of the presence of the target nucleic acid in the sample. In some embodiments, the detector 1508 may perform step 1608.

FIG. 16B shows an example process flow 1650 for a method of determining the concentration of a target nucleic acid in a sample, according to some embodiments. Process flow 1650 may include step 1652, step 1654, and step 1656. Detection system 1500 may perform process flow 1600.

At step 1652, the system receives the sample comprising the target nucleic acid such that the sample is contacted with a sense oligonucleotide and a capture oligonucleotide that is immobilized to a surface on the substrate via a silane-containing perfluorinated linker to produce a surface-immobilized target nucleic acid, wherein the capture oligonucleotide comprises a capture domain that is complementary to a first region of the target nucleic acid, wherein the sense oligonucleotide is functionalized with a first member of a binding pair and comprises a target-binding domain that is complementary to a second region of the target nucleic acid, and wherein the first region of the target nucleic acid does not overlap with the second region of the target nucleic acid. In some embodiments, the substrate 1502 may perform step 1652. In some embodiments, the addition component 1506 may add one or more of the sample or the sense oligonucleotide to the substrate 1502.

At step 1654, the system receives a detectable molecule that is conjugated to a second member of the binding pair such that the surface-immobilized target nucleic acid is contacted with the detectable molecule. In some embodiments, the substrate 1502 may perform step 1654. In some embodiments, the addition component 1506 may add the detectable molecule to the substrate 1502 such that it is contacted with the surface-immobilized target nucleic acid.

At step 1656, the system determines the concentration of the target nucleic acid based on detection of the detectable molecule. In some embodiments, the detector 1508 and/or the processor 1510 may perform step 1656. In some embodiments, the detector 1508 may detect the detectable molecule.

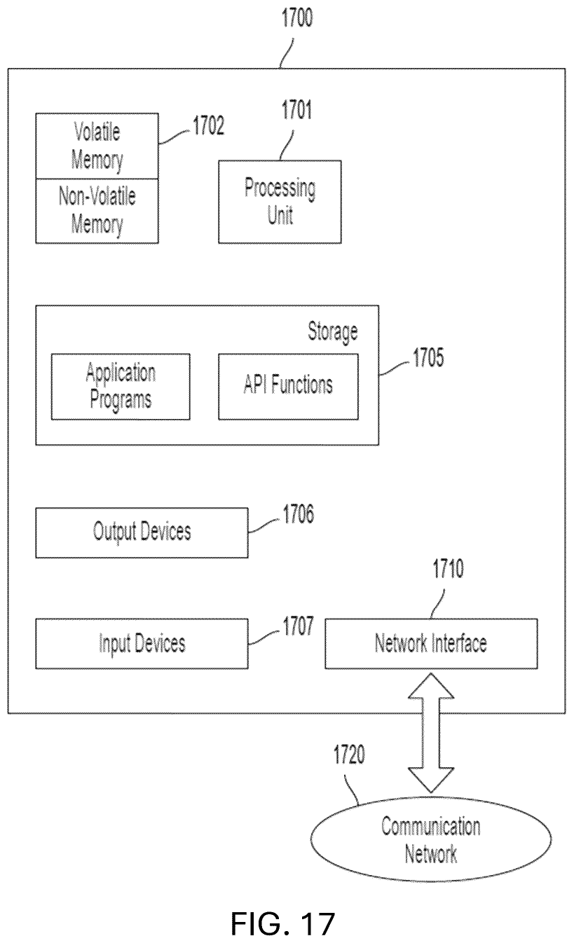

FIG. 17 shows an exemplary block diagram of a special purpose computing system that may implement and/or execute methods discussed herein. In the embodiment shown in FIG. 17, the computing system 1700 includes a processing unit 1701 having one or more processors and a non-transitory computer-readable storage medium 1702 that may include, for example, volatile and/or non-volatile memory. The non-transitory computer-readable storage medium 1702 may store one or more instructions to program the processing unit 1701 to perform any of the functions described herein. The computing system 1700 may also include other types of non-transitory computer-readable medium, such as storage 1705 (e.g., one or more disk drives) in addition to the non-transitory computer-readable storage medium 1702. The storage 1705 may also store one or more application programs and/or resources used by application programs (e.g., software libraries, or firmware), which may be loaded into the memory of non-transitory computer-readable storage medium 1702.

The computing system 1700 may have one or more input devices and/or output devices, such as devices 1706 and 1707 illustrated in FIG. 17. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, the input devices 1707 may include a microphone for capturing audio signals, and the output devices 1706 may include a display screen for visually rendering, and/or a speaker for audibly rendering, recognized text.

As shown in FIG. 17, the computing system 1700 may also comprise one or more network interfaces (e.g., the network interface 1710) to enable communication via various networks (e.g., the network 1720). Examples of networks include a local area network or a wide area network, such as an enterprise network or the Internet. Such networks may be based on any suitable technology and may operate according to any suitable protocol and may include wireless networks, wired networks or fiber optic networks. Such networks may include analog and/or digital networks.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. A method of detecting a target nucleic acid in a sample comprising:
(i) contacting a sample in a reaction mixture with
(a) a capture oligonucleotide that is immobilized to a surface via a silane-containing perfluorinated linker, wherein the capture oligonucleotide comprises a capture domain that is complementary to a first region of the target nucleic acid, and
(b) a sense oligonucleotide that is functionalized with a first member of a binding pair and comprises a target-binding domain that is complementary to a second region of the target nucleic acid, wherein the first region of the target nucleic acid does not overlap with the second region of the target nucleic acid, and
(ii) optionally removing nucleic acids that are not bound to the capture oligonucleotide from the reaction mixture,
(iii) adding a detectable molecule that is conjugated to a second member of the binding pair to the reaction mixture, and
(iv) detecting signal from surface-bound detectable label, wherein surface-bound signal at a level above background noise is indicative of the presence of the target nucleic acid in the sample.

2. A method of determining the concentration of a target nucleic acid in a sample, the method comprising:
(i) contacting a sample comprising the target nucleic acid with a sense oligonucleotide and a capture oligonucleotide that is immobilized to a surface via a silane-containing perfluorinated linker to produce a surface-immobilized target nucleic acid, wherein the capture oligonucleotide comprises a capture domain that is complementary to a first region of the target nucleic acid, wherein the sense oligonucleotide is functionalized with a first member of a binding pair and comprises a target-binding domain that is complementary to a second region of the target nucleic acid, and wherein the first region of the target nucleic acid does not overlap with the second region of the target nucleic acid;
(ii) contacting the surface-immobilized target nucleic acid with a detectable molecule that is conjugated to a second member of the binding pair; and
(iii) determining the concentration of the target nucleic acid based on detection of the detectable molecule.

3. The method of claim 1, wherein the target nucleic acid is a ribonucleic acid (RNA).

4. The method of claim 1, wherein the capture domain and/or the target-binding domain comprises one or more locked nucleotides (LNAs).

5. The method of claim 1, wherein the silane-containing perfluorinated linker comprises the structure of Formula (I):

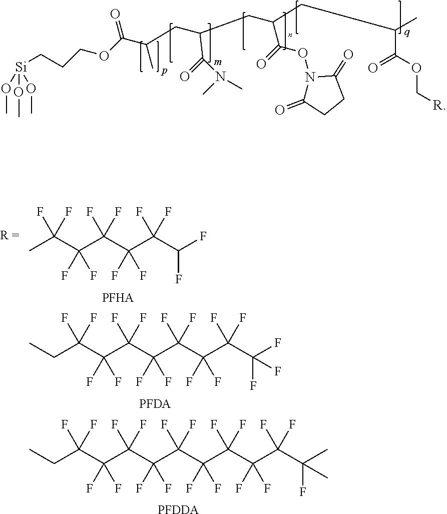

6. The method of claim 1, wherein the surface is a glass surface.

7. The method of claim 1, wherein the detectable molecule is a fluorescent molecule.

8. The method of claim 7, wherein the fluorescent molecule is a fluorophore or a quantum dot.

9. The method of claim 1, wherein the first member of the binding pair is biotin and/or the second member of the binding pair is streptavidin.

10. A system for detecting a target nucleic acid in a sample, the system comprising:
a substrate configured to receive a sample such that the sample is contacted in a reaction mixture with
(a) a capture oligonucleotide that is immobilized to a surface on the substrate via a silane-containing perfluorinated linker, wherein the capture oligonucleotide comprises a capture domain that is complementary to a first region of the target nucleic acid, and
(b) a sense oligonucleotide that is functionalized with a first member of a binding pair and comprises a target-binding domain that is complementary to a second region of the target nucleic acid, wherein the first region of the target nucleic acid does not overlap with the second region of the target nucleic acid,
the substrate further configured to receive a detectable molecule that is conjugated to a second member of the binding pair such that the detectable molecule is added to the reaction mixture, and
a detector configured to detect signal from surface-bound detectable label, wherein surface-bound signal at a level above background noise is indicative of the presence of the target nucleic acid in the sample.

11. The system of claim 10, wherein the detector comprises an image sensor.

12. The system of claim 10, further comprising:
a removal component configured to remove nucleic acids that are not bound to the capture oligonucleotide from the reaction mixture.

13. A system for determining the concentration of a target nucleic acid in a sample, the system comprising:
a substrate configured to receive the sample comprising the target nucleic acid such that the sample is contacted with a sense oligonucleotide and a capture oligonucleotide that is immobilized to a surface on the substrate via a silane-containing perfluorinated linker to produce a surface-immobilized target nucleic acid, wherein the capture oligonucleotide comprises a capture domain that is complementary to a first region of the target nucleic acid, wherein the sense oligonucleotide is functionalized with a first member of a binding pair and comprises a target-binding domain that is complementary to a second region of the target nucleic acid, and wherein the first region of the target nucleic acid does not overlap with the second region of the target nucleic acid,
the substrate further configured to receive a detectable molecule that is conjugated to a second member of the binding pair such that the surface-immobilized target nucleic acid is contacted with the detectable molecule; and
a processor configured to determine the concentration of the target nucleic acid based on detection of the detectable molecule.

14. The system of claim 10, wherein the target nucleic acid is a ribonucleic acid (RNA).

15. The system of claim 10, wherein the capture domain and/or the target-binding domain comprises one or more locked nucleotides (LNAs).

16. The system of claim 10, wherein the silane-containing perfluorinated linker comprises the structure of Formula (I):

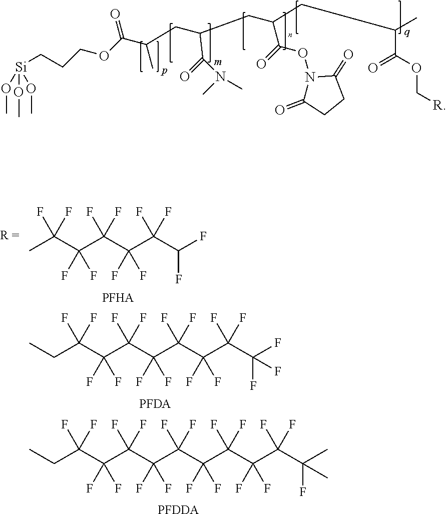

17. The system of claim 10, wherein the surface is a glass surface.

18. The system of claim 10, wherein the detectable molecule is a fluorescent molecule.

19. The system of claim 18, wherein the fluorescent molecule is a fluorophore or a quantum dot.

20. The system of claim 10, wherein the first member of the binding pair is biotin and/or the second member of the binding pair is streptavidin.


SEQUENCE LISTING (9 sequences)

SEQ ID NO: 1 - DNA, 33 bp, synthetic construct
  source 1..33: mol_type = other DNA
  modified_base order(3,6,10,19,23,29,32): mod_base = OTHER; note = locked nucleic acids
  modified_base 33: mod_base = OTHER; note = 3 prime dideoxy-cytosine
  misc_binding <1: bound_moiety = 5 prime amine spaced by 6 methylenes
taccgcgtgtacatagtacacacccgcagcttc

SEQ ID NO: 2 - DNA, 17 bp, synthetic construct
  source 1..17: mol_type = other DNA
  modified_base order(5,8,10,12,14,16): mod_base = OTHER; note = locked nucleic acids
  modified_base 11: mod_base = OTHER; note = deoxythymidine modified by internal biotin
  misc_binding <1: bound_moiety = 2 biotins linked to 5 prime by amides
  misc_binding >17: bound_moiety = 3 prime biotin spaced by triethylene glycol (TEG)
ggtttatcgcttagccc

SEQ ID NO: 3 - DNA, 32 bp, synthetic construct
  source 1..32: mol_type = other DNA
  misc_binding <1: bound_moiety = 5 prime amine spaced by 6 methylenes
  modified_base 32: mod_base = OTHER; note = 3 prime dideoxy-cytosine
  modified_base order(3,7,14,18,23,29,31): mod_base = OTHER; note = locked nucleic acids
tcctgtccgtgtccgggccgggtgaggtttcc

SEQ ID NO: 4 - DNA, 18 bp, synthetic construct
  source 1..18: mol_type = other DNA
  modified_base order(3..7,9,11,15): mod_base = OTHER; note = locked nucleic acids
  modified_base 10: mod_base = OTHER; note = deoxythymidine modified by internal biotin
  misc_binding <1: bound_moiety = 2 biotins linked to 5 prime by amides
  misc_binding >18: bound_moiety = 3 prime biotin spaced by triethylene glycol (TEG)
cgtgttgagtcaaattaa

SEQ ID NO: 5 - DNA, 40 bp, synthetic construct
  source 1..40: mol_type = other DNA
  misc_binding <1: bound_moiety = 5 prime amine spaced by 6 methylenes
caggtagatrttgaaagatgagycttctaaccgaggtcga

SEQ ID NO: 6 - DNA, 41 bp, synthetic construct
  source 1..41: mol_type = other DNA
  modified_base order(3,8,13,17,19,25): mod_base = OTHER; note = locked nucleic acids
  misc_binding >41: bound_moiety = 3 prime amine spaced by 6 methylenes
ttcttccccaaatcggcaccaatgctatttttttttttttt

SEQ ID NO: 7 - DNA, 18 bp, synthetic construct
  source 1..18: mol_type = other DNA
  modified_base 11: mod_base = OTHER; note = fluorescein attached to position 5 of the thymine ring by a 6-carbon spacer arm
  modified_base order(3,9..10,13..14,16): mod_base = OTHER; note = locked nucleic acids
  misc_binding <1: bound_moiety = 2 biotins linked to 5 prime by amides
cttgcatgcctttagcca

SEQ ID NO: 8 - DNA, 25 bp, synthetic construct
  source 1..25: mol_type = other DNA
  modified_base order(4,7,10,14,20,24): mod_base = OTHER; note = locked nucleic acids
  misc_binding <1: bound_moiety = 5 prime amine spaced by 6 methylenes
aggttactttgtaagcctgtgaacg

SEQ ID NO: 9 - DNA, 18 bp, synthetic construct
  source 1..18: mol_type = other DNA
  modified_base order(3,6,9,12,15,17): mod_base = OTHER; note = locked nucleic acids
  misc_binding <1: bound_moiety = 2 biotins linked to 5 prime by amides
  modified_base 10: mod_base = OTHER; note = fluorescein attached to position 5 of the thymine ring by a 6-carbon spacer arm
gcgcagagctctgacgaa